(19) United States
(12) Patent Application Publication
(10) Pub. No.: US 2026/0126438 A1
(43) Pub. Date: May 7, 2026

(54) SYSTEMS AND METHOD FOR TARGET ENGAGEMENT

(71) Applicant: Promega Corporation, Madison, WI (US)

(72) Inventors: Cesear CORONA, Madison, WI (US); Ming CHEN, Madison, WI (US); Wenhui ZHOU, Madison, WI (US)

(21) Appl. No.: 19/295,373

(22) Filed: Aug. 8, 2025

Related U.S. Application Data
(60) Provisional application No. 63/681,677, filed on Aug. 9, 2024.
(60) Provisional application No. 63/688,120, filed on Aug. 28, 2024.

(51) Int. Cl. G01N 33/543; C12N 9/02; C12N 9/14; C12Q 1/34; C12Q 1/66; G01N 21/64; G01N 21/76; G01N 33/68

(52) U.S. Cl. CPC G01N 33/54373; C12N 9/0069; C12N 9/14; C12Q 1/34; C12Q 1/66; G01N 21/6428; G01N 21/763; G01N 33/6803; C07K 2319/61; C12Y 113/12013; C12Y 308/01005; G01N 2021/6432; G01N 2333/90241; G01N 2333/914

(57) ABSTRACT

Provided herein are systems and methods for the detection and quantification of a target protein via bioluminescence and/or bioluminescent resonance energy transfer (BRET). In particular, provided herein are assay methods using a multi-component biochemical system that assembles into a detectable in vitro chemical complex at a protein target of interest.

### CROSS-REFERENCE

This application claims the benefit of U.S. Provisional Patent Application No. 63/681,677, filed Aug. 9, 2024, and Provisional Patent Application No. 63/688,120, filed Aug. 28, 2024; each of which is incorporated by reference herein in its entirety.

### SEQUENCE LISTING PARAGRAPH

The text of the computer readable sequence listing filed herewith, titled “PRMG_43257.203_SequenceListing.xml”, created Sep. 12, 2025, having a file size of 8,886 bytes, is hereby incorporated by reference in its entirety.

### FIELD

Provided herein are systems and methods for the detection and quantification of a target protein via bioluminescence and/or bioluminescent resonance energy transfer (BRET). In particular, provided herein are assay methods using a multi-component biochemical system that assembles into a detectable in vitro chemical complex at a protein target of interest.

### BACKGROUND

Identification of protein binding compounds is a fundamental need in drug discovery. These binding compounds are often identified through established screening methods that require a degree of specificity, sensitivity, and robustness to support high throughput. Current assay methods are reliant on the binding of a manageable subset members of a compound library binding with sufficient affinity to be detected, isolated, and identified. Often, especially in the case of challenging or “intractable” targets, identified hit compounds function sufficiently to serve as tool compounds but fail to mature into viable clinical candidates, leading to a shortage of chemical scaffolds for future medicinal chemistry efforts.

### SUMMARY

Provided herein are systems and methods for the detection and quantification of a target protein via bioluminescence and/or bioluminescent resonance energy transfer (BRET). In particular, provided herein are assay methods using a multi-component biochemical system that assembles into a detectable in vitro chemical complex at a protein target of interest.

In some embodiments, provided herein are systems comprising: (a) a bioluminescent protein or a bioluminescent complex tethered to a target protein; (b) fluorophore tethered to a reference ligand for the target protein; (c) a test ligand for the target protein; and (d) a substrate for the bioluminescent protein or complex; wherein the bioluminescent protein or complex emits a bioluminescent signal at a first wavelength in the presence of the substrate; wherein the fluorophore absorbs light at the first wavelength and emits a fluorescent signal at the second wavelength; wherein when the reference ligand is bound to the target protein the fluorophore is in proximity to the bioluminescent protein or complex to allow bioluminescence resonance energy transfer (BRET) from the bioluminescent protein or complex; and wherein if the test ligand is capable of binding to the target protein the test ligand competes with the reference ligand for binding to the target protein, thereby decreasing the fluorescent signal resulting from the BRET.

In some embodiments, the bioluminescent protein or a bioluminescent complex tethered to a target protein is a bioluminescent protein. In some embodiments, the bioluminescent protein is a luciferase. In some embodiments, the luciferase is an Oplophorous-derived polypeptide that comprises at least 70% (e.g., 70%, 75%, 80%, 85%, 90%, 95%, 100%, or ranges therebetween) sequence identity with SEQ ID NO: 1. In some embodiments, the luciferase comprises 100% sequence identity with SEQ ID NO: 1. In some embodiments, the luciferase is a circularly permuted variant of an Oplophorous-derived polypeptide.

In some embodiments, the bioluminescent protein or a bioluminescent complex tethered to a target protein is a bioluminescent complex; wherein a first component of the bioluminescent complex is tethered to the target protein; wherein a second component of the bioluminescent complex is not tethered to the target protein. In some embodiments, the first and second components of the bioluminescent complex form an active bioluminescent complex upon binding to each other; and wherein the first and second components of the bioluminescent complex require facilitation to form an active bioluminescent complex. In some embodiments, the first and second components of the bioluminescent complex collectively comprise at least 70% (e.g., 70%, 75%, 80%, 85%, 90%, 95%, 100%, or ranges therebetween) sequence identity with SEQ ID NO: 1 or 2. In some embodiments, the first component of the bioluminescent complex comprises at least 70% (e.g., 70%, 75%, 80%, 85%, 90%, 95%, 100%, or ranges therebetween) sequence identity with SEQ ID NO: 3. In some embodiments, the first component of the bioluminescent complex has 100% sequence identity with SEQ ID NO: 3. In some embodiments, the second component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 5.

In some embodiments, the bioluminescent protein or complex is fused to the target protein directly or by a linker peptide/polypeptide. In some embodiments, the bioluminescent protein or complex is tethered to an affinity molecule capable of binding to the target protein or an affinity tag thereon. In some embodiments, the affinity molecule is an antibody or antibody fragment, and the target protein is tethered to an affinity tag to which the antibody or antibody fragment is capable of binding. In some embodiments, the bioluminescent protein or complex is fused a capture agent capable of covalently binding to a capture ligand tethered to the target protein. In some embodiments, the capture agent is a modified dehalogenase capable of forming a covalent bond to a haloalkyl moiety and the capture ligand is a haloalkyl moiety. In some embodiments, the modified dehalogenase comprises at least 70% (e.g., 70%, 75%, 80%, 85%, 90%, 95%, 100%, or ranges therebetween) sequence identity to SEQ ID NO: 8. In some embodiments, the substrate for the bioluminescent protein or complex is a coelenterazine molecule. In some embodiments, the coelenterazine molecule is a furimazine molecule. In some embodiments, the furimazine molecule is furimazine or fluorofurimazine. In some embodiments, the fluorophore is a is a rhodamine dye. In some embodiments, the rhodamine dye is selected from:

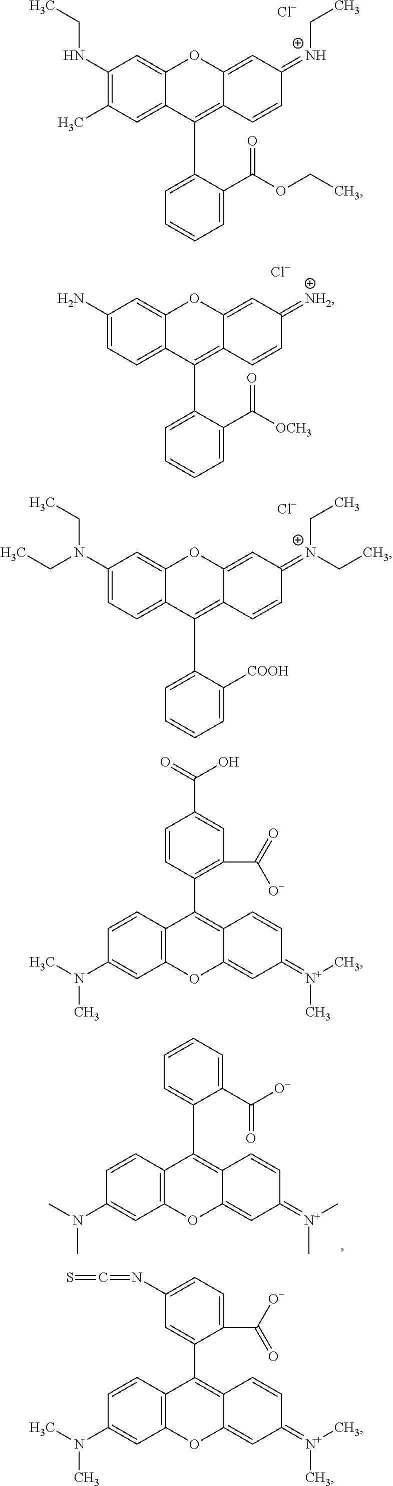

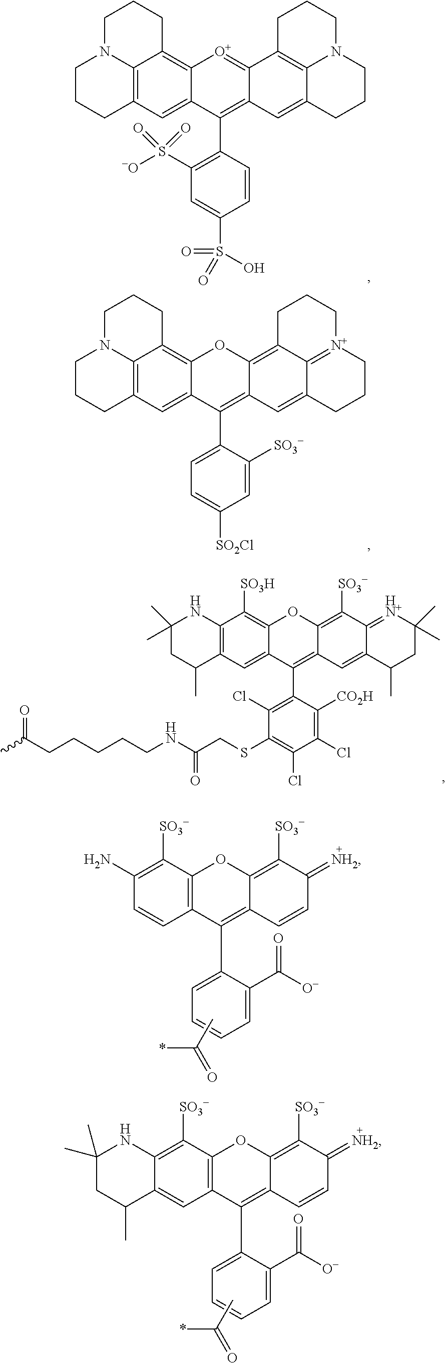

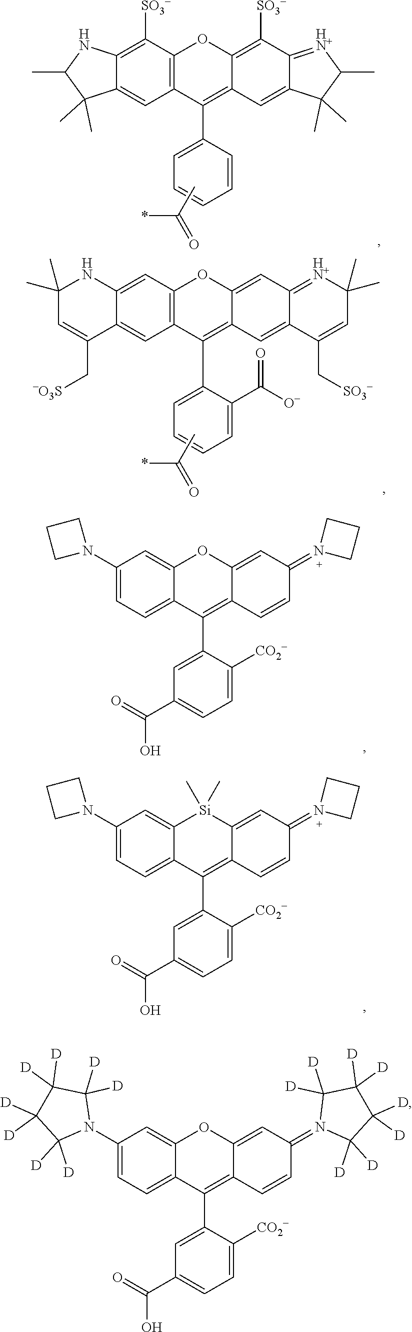

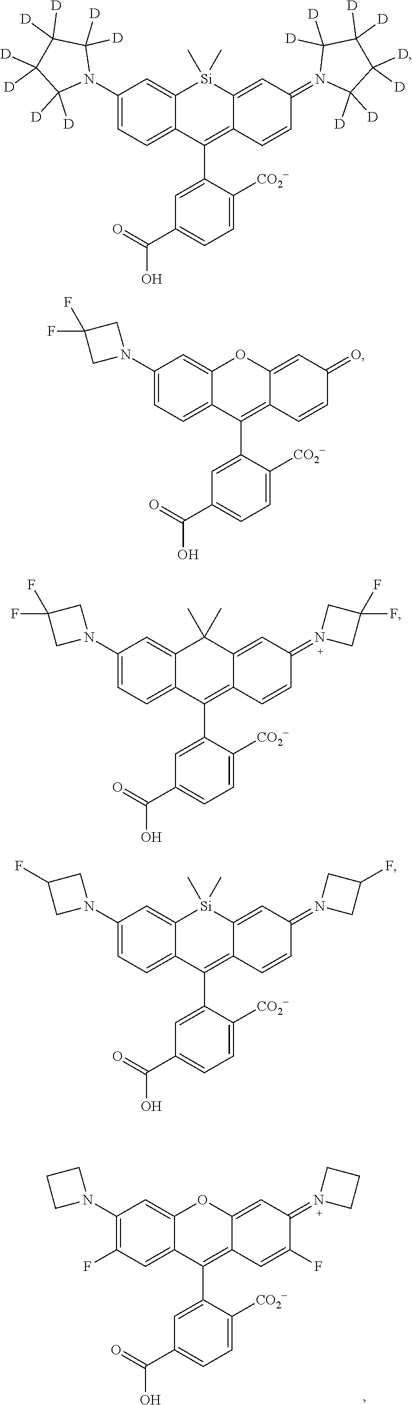

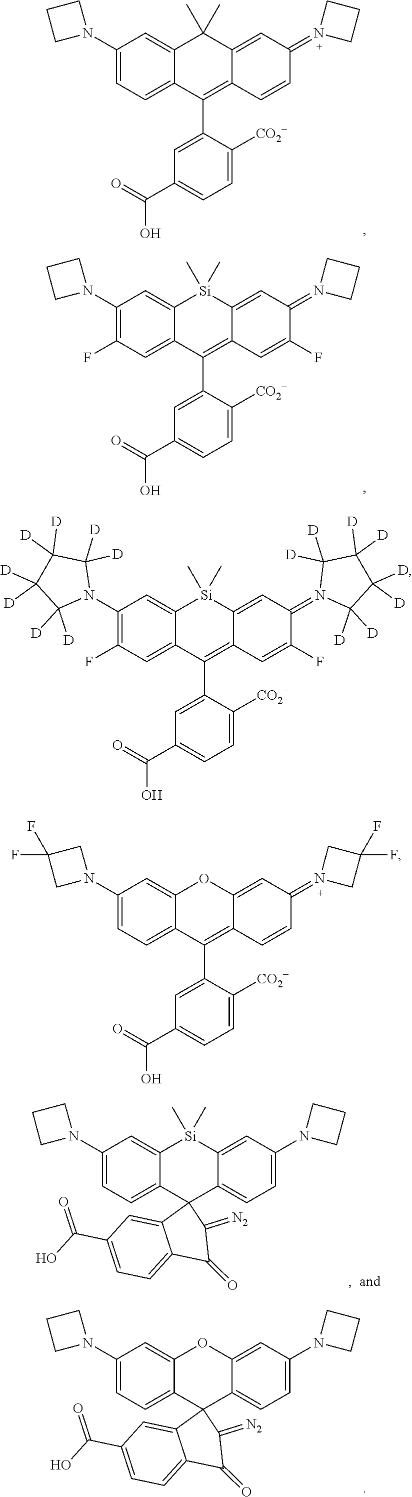

In some embodiments, provided herein are systems comprising: (a) a first component of a bioluminescent complex tethered to a target protein; (b) a second component of the bioluminescent complex tethered to a reference ligand; (c) a test ligand for the target protein; and (d) a substrate for the bioluminescent protein or complex; wherein the bioluminescent protein or complex emits a bioluminescent signal in the presence of the substrate; wherein when the reference ligand is bound to the target protein the second component of the bioluminescent complex is in proximity to the first component of the bioluminescent complex to facilitate formation the bioluminescent complex and generation of the bioluminescent signal in the presence of the substrate; and wherein if the test ligand is capable of binding to the target protein the test ligand competes with the reference ligand for binding to the target protein, thereby decreasing the bioluminescent signal from the bioluminescent complex. In some embodiments, the first and second components of the bioluminescent complex form an active bioluminescent complex upon binding to each other. In some embodiments, first and second components of the bioluminescent complex collectively comprise at least 70% (e.g., 60%, 65%, 70%, 75%, 80%, 85%, 90%, 95%, 96%, 97%, 98%, 99%, 100%, or ranges therebetween) sequence identity with SEQ ID NO: 1 or 2. In some embodiments, the second component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 4. In some embodiments, the second component of the bioluminescent complex has 100% sequence identity with SEQ ID NO: 4. In some embodiments, the first component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 5. In some embodiments, the first component of the bioluminescent complex has 100% sequence identity with SEQ ID NO: 5. In some embodiments, the first component of the bioluminescent complex is fused to the target protein directly or by a linker peptide/polypeptide. In some embodiments, the first component of the bioluminescent complex is tethered to an affinity molecule capable of binding to the target protein or an affinity tag thereon. In some embodiments, the affinity molecule is an antibody or antibody fragment, and the target protein is tethered to an affinity tag to which the antibody or antibody fragment is capable of binding. In some embodiments, the first component of the bioluminescent complex is fused a capture agent capable of covalently binding to a capture ligand tethered to the target protein. In some embodiments, the capture agent is a modified dehalogenase capable of forming a covalent bond to a haloalkyl moiety and the capture ligand is a haloalkyl moiety. In some embodiments, the modified dehalogenase comprises at least 70% (e.g., 60%, 65%, 70%, 75%, 80%, 85%, 90%, 95%, 96%, 97%, 98%, 99%, 100%, or ranges therebetween) sequence identity to SEQ ID NO: 8. In some embodiments, the substrate for the bioluminescent protein or complex is a coelenterazine molecule. In some embodiments, the coelenterazine molecule is a furimazine molecule. In some embodiments, the furimazine molecule is furimazine or fluorofurimazine.

In some embodiments, provided herein are methods comprising: (a) contacting within a biochemical sample: (i) a bioluminescent protein or a bioluminescent complex tethered to a target protein; (ii) fluorophore tethered to a reference ligand for the target protein; and (iii) a substrate for the bioluminescent protein or complex; wherein the bioluminescent protein or complex emits a bioluminescent signal at a first wavelength in the presence of the substrate; wherein the fluorophore absorbs light at the first wavelength and emits a fluorescent signal at the second wavelength; wherein when the reference ligand is bound to the target protein the fluorophore is in proximity to the bioluminescent protein or complex to allow bioluminescence resonance energy transfer (BRET) from the bioluminescent protein or complex; (b) detecting bioluminescence from the bioluminescent protein or complex; (c) contacting the sample with a test ligand for the target protein; and (d) detecting bioluminescence from the bioluminescent protein or complex; wherein if the test ligand is capable of binding to the target protein the test ligand competes with the reference ligand for binding to the target protein, thereby decreasing the fluorescent signal resulting from the BRET.

In some embodiments, the bioluminescent protein or a bioluminescent complex tethered to a target protein is a bioluminescent protein. In some embodiments, the bioluminescent protein is a luciferase. In some embodiments, the luciferase is an Oplophorous-derived polypeptide that comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, the luciferase comprises 100% sequence identity with SEQ ID NO: 1. In some embodiments, the luciferase is a circularly permuted variant of an Oplophorous-derived polypeptide.

In some embodiments, the bioluminescent protein or a bioluminescent complex tethered to a target protein is a bioluminescent complex; wherein a first component of the bioluminescent complex is tethered to the target protein; wherein a second component of the bioluminescent complex is not tethered to the target protein. In some embodiments, the first and second components of the bioluminescent complex form an active bioluminescent complex upon binding to each other; and wherein the first and second components of the bioluminescent complex require facilitation to form an active bioluminescent complex. In some embodiments, first and second components of the bioluminescent complex collectively comprise at least 70% (e.g., 60%, 65%, 70%, 75%, 80%, 85%, 90%, 95%, 96%, 97%, 98%, 99%, 100%, or ranges therebetween) sequence identity with SEQ ID NO: 1 or 2. In some embodiments, the first component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 3. In some embodiments, the first component of the bioluminescent complex has 100% sequence identity with SEQ ID NO: 3. In some embodiments, the second component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 5.

In some embodiments, the bioluminescent protein or complex is fused to the target protein directly or by a linker peptide/polypeptide. In some embodiments, the bioluminescent protein or complex is tethered to an affinity molecule capable of binding to the target protein or an affinity tag thereon. In some embodiments, In some embodiments, the affinity molecule is an antibody or antibody fragment, and the target protein is tethered to an affinity tag to which the antibody or antibody fragment is capable of binding. In some embodiments, the bioluminescent protein or complex is fused a capture agent capable of covalently binding to a capture ligand tethered to the target protein. In some embodiments, the capture agent is a modified dehalogenase capable of forming a covalent bond to a haloalkyl moiety and the capture ligand is a haloalkyl moiety. In some embodiments, the modified dehalogenase comprises at least 70% (e.g., 60%, 65%, 70%, 75%, 80%, 85%, 90%, 95%, 96%, 97%, 98%, 99%, 100%, or ranges therebetween) sequence identity to SEQ ID NO: 8. In some embodiments, the substrate for the bioluminescent protein or complex is a coelenterazine molecule. In some embodiments, the coelenterazine molecule is a furimazine molecule. In some embodiments, the furimazine molecule is furimazine or fluorofurimazine. In some embodiments, the fluorophore is a is a rhodamine dye. In some embodiments, the rhodamine dye is selected from:

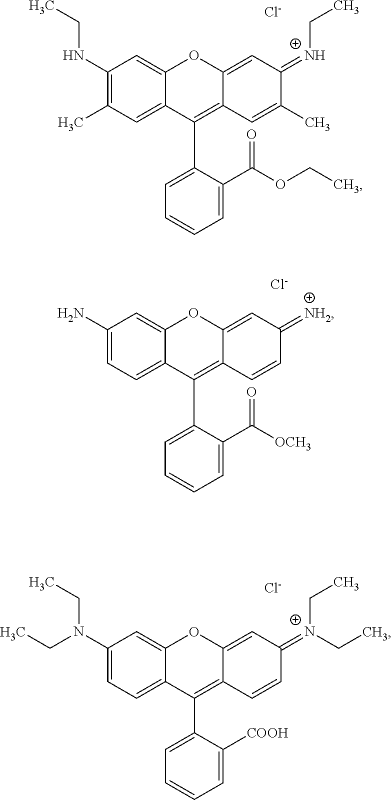

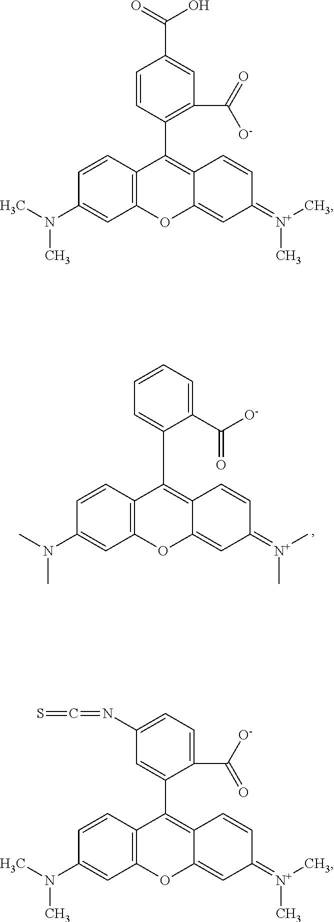

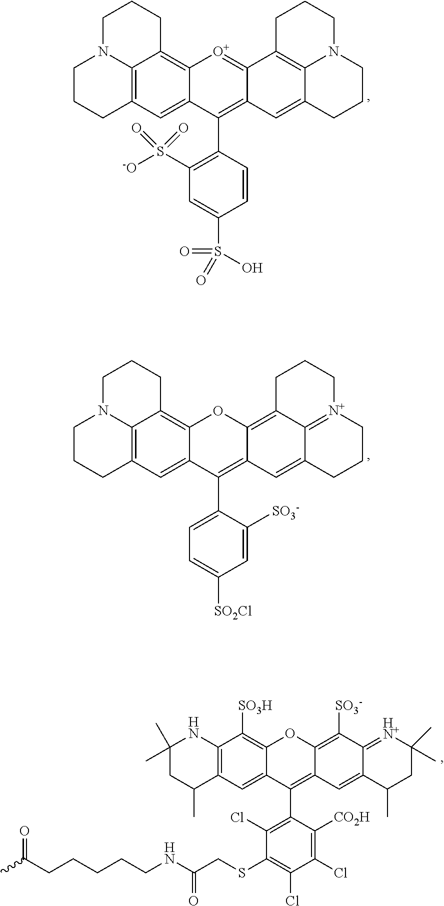

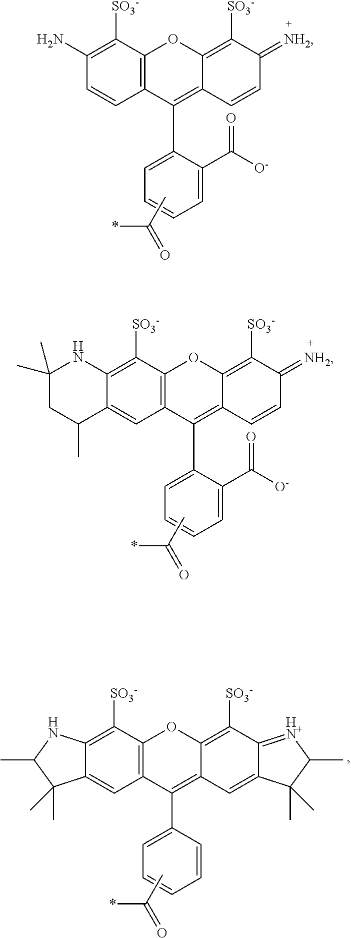

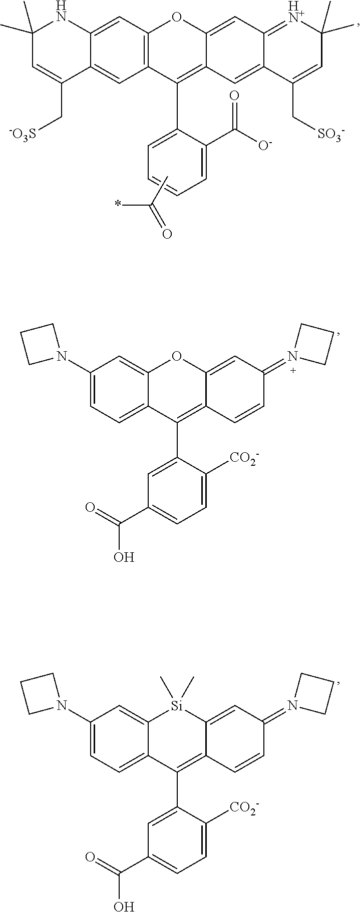

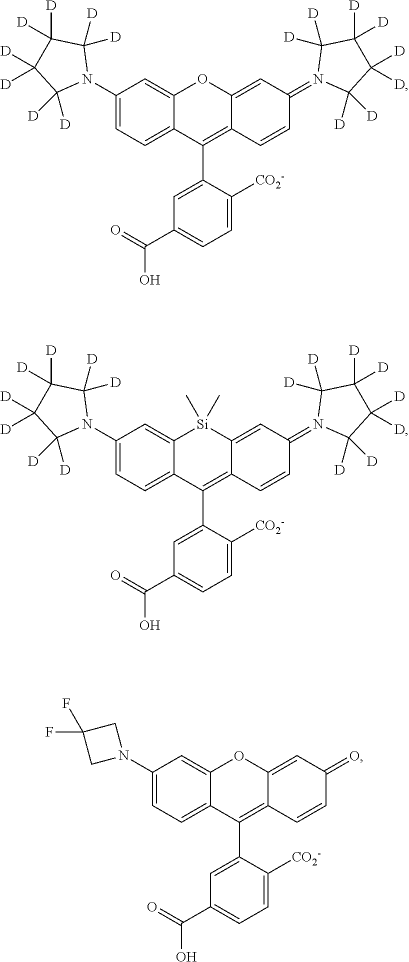

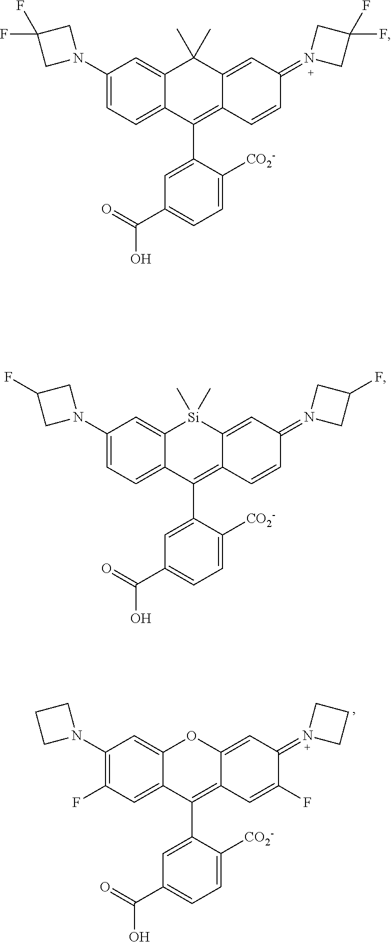

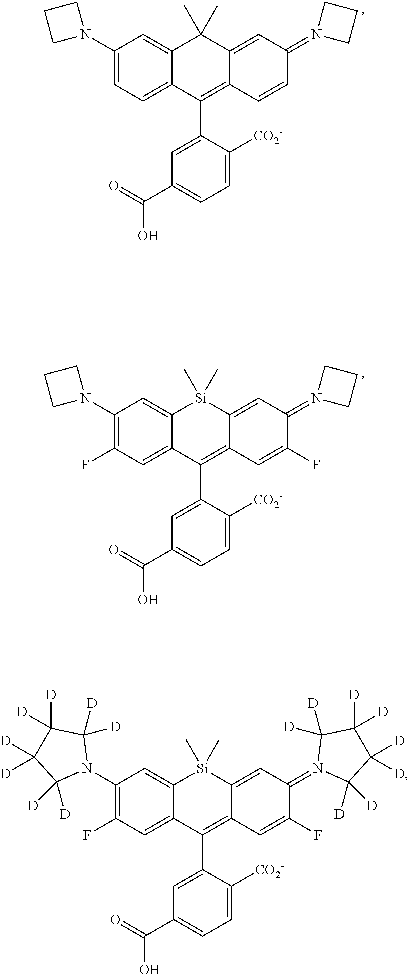

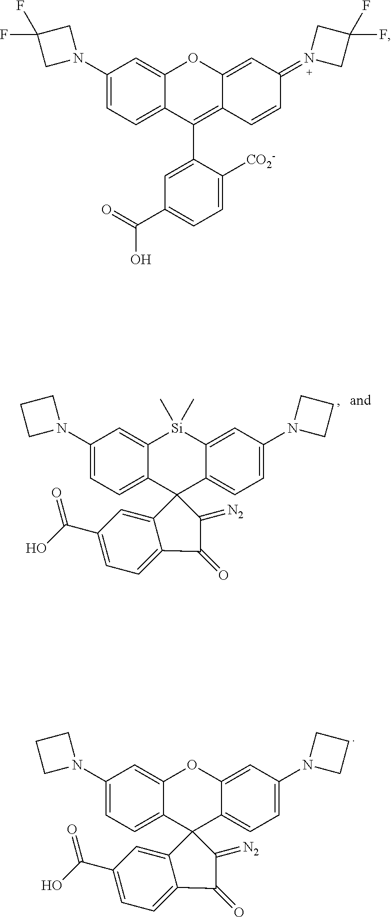

In some embodiments, provided herein are methods comprising: (a) a first component of a bioluminescent complex tethered to a target protein; (b) a second component of the bioluminescent complex tethered to a reference ligand; (c) a test ligand for the target protein; and (d) a substrate for the bioluminescent protein or complex; wherein the bioluminescent protein or complex emits a bioluminescent signal in the presence of the substrate; wherein when the reference ligand is bound to the target protein the second component of the bioluminescent complex is in proximity to the first component of the bioluminescent complex to facilitate formation the bioluminescent complex and generation of the bioluminescent signal in the presence of the substrate; and wherein if the test ligand is capable of binding to the target protein the test ligand competes with the reference ligand for binding to the target protein, thereby decreasing the bioluminescent signal from the bioluminescent complex. In some embodiments, the first and second components of the bioluminescent complex form an active bioluminescent complex upon binding to each other. In some embodiments, the first and second components of the bioluminescent complex collectively comprise at least 70% (e.g., 60%, 65%, 70%, 75%, 80%, 85%, 90%, 95%, 96%, 97%, 98%, 99%, 100%, or ranges therebetween) sequence identity with SEQ ID NO: 1 or 2. In some embodiments, the second component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 4. In some embodiments, the second component of the bioluminescent complex has 100% sequence identity with SEQ ID NO: 4. In some embodiments, the first component of the bioluminescent complex comprises at least 70% (e.g., 60%, 65%, 70%, 75%, 80%, 85%, 90%, 95%, 96%, 97%, 98%, 99%, 100%, or ranges therebetween) sequence identity with SEQ ID NO: 5. In some embodiments, the first component of the bioluminescent complex has 100% sequence identity with SEQ ID NO: 5. In some embodiments, the first component of the bioluminescent complex is fused to the target protein directly or by a linker peptide/polypeptide. In some embodiments, the first component of the bioluminescent complex is tethered to an affinity molecule capable of binding to the target protein or an affinity tag thereon. In some embodiments, the affinity molecule is an antibody or antibody fragment, and the target protein is tethered to an affinity tag to which the antibody or antibody fragment is capable of binding. In some embodiments, the first component of the bioluminescent complex is fused a capture agent capable of covalently binding to a capture ligand tethered to the target protein. In some embodiments, the capture agent is a modified dehalogenase capable of forming a covalent bond to a haloalkyl moiety and the capture ligand is a haloalkyl moiety. In some embodiments, the modified dehalogenase comprises at least 70% % (e.g., 60%, 65%, 70%, 75%, 80%, 85%, 90%, 95%, 96%, 97%, 98%, 99%, 100%, or ranges therebetween) sequence identity to SEQ ID NO: 8. In some embodiments, the substrate for the bioluminescent protein or complex is a coelenterazine molecule. In some embodiments, the coelenterazine molecule is a furimazine molecule. In some embodiments, the furimazine molecule is furimazine or fluorofurimazine.

### BRIEF DESCRIPTION OF THE DRAWINGS

FIG. 1A-E. Cartoon depictions of exemplary embodiments of target engagement assays within the scope herein (A) a component of bioluminescent complex is tethered to a protein of interest (POI) by an antibody/affinity tag interaction; binding of a reference ligand to the POI is required for formation of a bioluminescent complex; binding of a test ligand to the POI results in loss of bioluminescent signal; (B) a component of bioluminescent complex is tethered to a POI by direct fusion; binding of a reference ligand to the POI is required for formation of a bioluminescent complex; binding of a test ligand to the POI results in loss of bioluminescent signal; (C) a component of bioluminescent complex is tethered to a POI by a HALOTAG/HT ligand covalent interaction; binding of a reference ligand to the POI is required for formation of a bioluminescent complex; binding of a test ligand to the POI results in loss of bioluminescent signal; (D) a bioluminescent protein is tethered to a POI and a reference ligand is tethered to a fluorophore; BRET from the bioluminescent protein to the fluorophore occurs upon binding of the reference ligand to the POI; binding of a test ligand to the POI results in loss in BRET signal; (E) a first component of bioluminescent complex is tethered to a POI and a second component of the bioluminescent complex with high affinity for the first component is included in the system; a reference ligand is tethered to a fluorophore; BRET from the bioluminescent complex to the fluorophore occurs upon binding of the reference ligand to the POI; binding of a test ligand to the POI results in loss in BRET signal; and (F) a component of bioluminescent complex is tethered to a secondary antibody or antibody fragment capable of binding to a primary antibody or antibody fragment that is capable of binding to a protein of interest (POI) or an affinity tag tethered thereto; binding of a reference ligand to the POI is required for formation of a bioluminescent complex; binding of a test ligand to the POI results in loss of bioluminescent signal.

FIG. 2A-C. CDK8/Cyclin C-His Assay. (A) Plate map showing the titration matrix of CDK8/CyclinC and SmBiT-Tracer to determine optimal protein/tracer concentrations. (B) SmBiT-Tracer titration to determine the dissociation constant (Kd) of the probe. (C) Experimental determination of apparent IC50 of described CDK8 inhibitors.

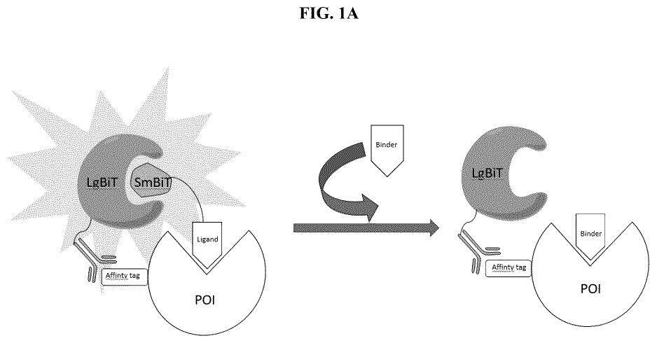

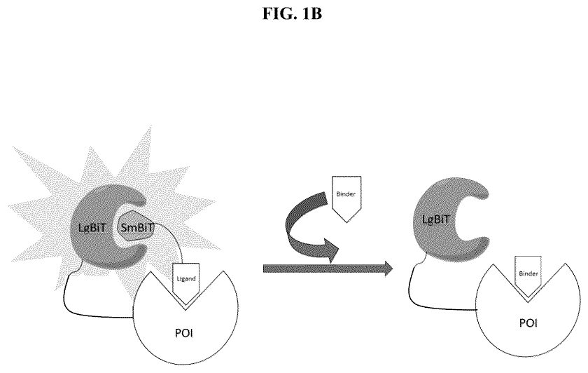

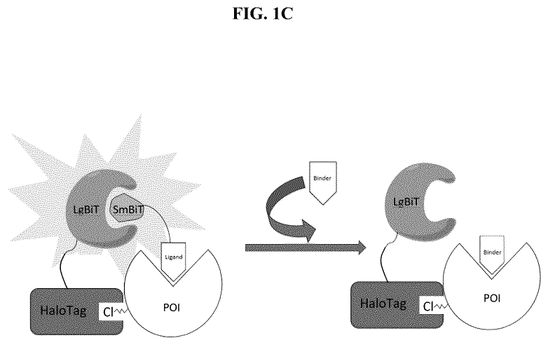

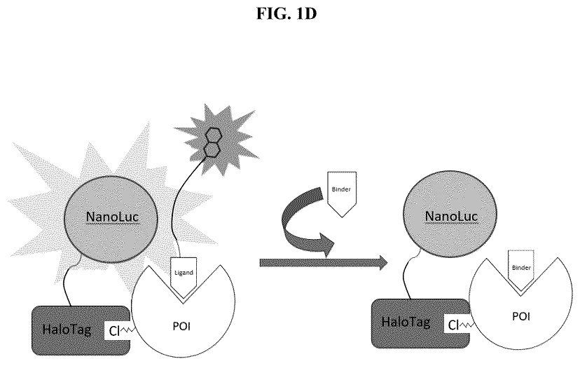

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a peptide” is a reference to one or more peptides and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “system” refers a group of devices, reagents, compositions, etc. that are collectively grouped for a desired function or objective. The components of the system may reside in a single reaction mixture, cell, container, etc. or may be maintained separately, e.g., for subsequent combination to achieve the desired function or objective.

As used herein, the term “substantially” means that the recited characteristic, parameter, and/or value need not be achieved exactly, but that deviations or variations, including for example, tolerances, measurement error, measurement accuracy limitations and other factors known to skill in the art, may occur in amounts that do not preclude the effect the characteristic was intended to provide. A characteristic or feature that is substantially absent (e.g., substantially non-luminescent) may be one that is within the noise, beneath background, below the detection capabilities of the assay being used, or a small fraction (e.g., <1%, <0.1%, <0.01%, <0.001%, <0.00001%, <0.000001%, <0.0000001%) of the significant characteristic (e.g., luminescent intensity of a bioluminescent protein or bioluminescent complex).

As used herein, the term “luminescence” refers to the emission of light by a substance as a result of a chemical reaction (“chemiluminescence”) or an enzymatic reaction (“bioluminescence”).

As used herein, the term “bioluminescence” refers to production and emission of light by a reaction catalyzed by, or enabled by, an enzyme, protein, protein complex, or other biomolecule (e.g., bioluminescent complex). In typical embodiments, a substrate for a bioluminescent entity (e.g., bioluminescent protein or bioluminescent complex) is converted into an unstable form by the bioluminescent entity; the substrate subsequently emits light.

As used herein, the term “luminophore” refers to a chemical moiety or compound that can be placed in an excited electronic state (e.g., by a chemical or enzymatic reaction) and emits light as it returns to its electronic ground state.

As used herein, the term “imidazopyrazine luminophore” refers to a genus of luminophores including “native coelenterazine” as well as synthetic (e.g., derivative or variant) and natural analogs thereof, including furimazine, furimazine analogs (e.g., fluorofurimazine) coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); U.S. Pat. No. 8,669,103; U.S. Prov. App. No. 63/379,573; the disclosures of which are incorporated by reference herein in their entireties.

As used herein, the term “coelenterazine” refers to the naturally-occurring (“native”) imidazopyrazine of the structure:

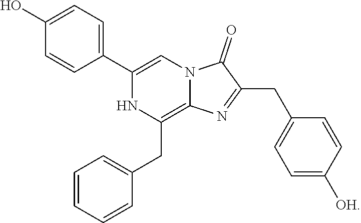 [SMILES: O=c1c(Cc2ccc(O)cc2)nc2c(Cc3ccccc3)[nH]c(-c3ccc(O)cc3)cn1-2]

As used herein, the term “furimazine” refers to the coelenterazine derivative of the structure:

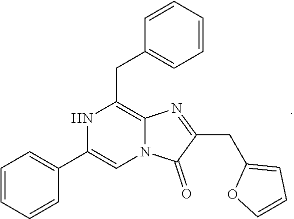 [SMILES: O=c1c(Cc2ccco2)nc2c(Cc3ccccc3)[nH]c(-c3ccccc3)cn1-2]

As used herein, the term “fluorofurimazine” refers to the furimazine derivative of the structure:

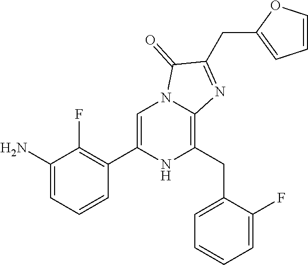 [SMILES: Nc1cccc(-c2cn3c(=O)c(Cc4ccco4)nc-3c(Cc3ccccc3F)[nH]2)c1F]

(U.S. application Ser. No. 16/548,214; incorporated by reference in its entirety).

As used herein, the term “bioluminescence resonance energy transfer” (“BRET”) refers to the distance-dependent interaction in which energy is transferred from a donor bioluminescent protein/complex and substrate to an acceptor molecule without emission of a photon. The efficiency of BRET is dependent on the inverse sixth power of the intermolecular separation, making it useful over distances comparable with the dimensions of biological macromolecules (e.g., within 30-80 Å, depending on the degree of spectral overlap).

As used herein, the term “an Oplophorus luciferase” (“an OgLuc”) refers to a luminescent polypeptide having significant sequence identity, structural conservation, and/or the functional activity of the luciferase produced by and derived from the deep-sea shrimp Oplophorus gracilirostris. In particular, an OgLuc polypeptide refers to a luminescent polypeptide having significant sequence identity, structural conservation, and/or the functional activity of the mature 19 kDa subunit of the Oplophorus luciferase protein complex (e.g., without a signal sequence) such as SEQ ID NOs: 1 (NANOLUC), which comprises 10 β strands (β1, β2, β3, β4, β5, β6, β7, β8, β9, β10) and utilize substrates such as coelenterazine or a coelenterazine derivative or analog to produce luminescence.

As used herein the term “complementary” refers to the characteristic of two or more structural elements (e.g., peptide, polypeptide, nucleic acid, small molecule, etc.) being able to hybridize, dimerize, or otherwise form a complex with each other. For example, a “complementary peptide and polypeptide” are capable of coming together to form a complex. Complementary elements may require assistance (facilitation) to form a complex (e.g., from interaction elements), for example, to place the elements in the proper conformation for complementarity, to co-localize complementary elements, to lower interaction energy for complementary, to overcome low affinity for one another, etc.

As used herein, the term “complex” refers to an assemblage or aggregate of molecules (e.g., peptides, polypeptides, etc.) in direct and/or indirect contact with one another. In one aspect, “contact,” or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such an aspect, a complex of molecules (e.g., peptides and polypeptide) is formed under assay conditions such that the complex is thermodynamically favored (e.g., compared to a non-aggregated, or non-complexed, state of its component molecules). As used herein, the term “complex,” unless described as otherwise, refers to the assemblage of two or more molecules (e.g., peptides, polypeptides, or a combination thereof).

As used herein, the terms “conjugated” and “conjugation” refer to the covalent attachment of two molecular entities (e.g., post-synthesis and/or during synthetic production). Conjugated entities may be peptides or proteins that are “fused” by a peptide linkage, or may also include other molecular entities (e.g., nucleic acid, small molecules, etc.) connected directly or by suitable linkers.

As used herein, the term “capture protein” or “capture agent” refers to a protein or other molecular entity that forms a stable covalent bond with its substrate, ligand, or other molecular element upon interaction therewith. A capture protein may be a receptor that forms a covalent bond upon binding its ligand or an enzyme that forms a covalent bond with its substrate. An example of a suitable capture protein for use in embodiments of the present invention is the HALOTAG protein described in U.S. Pat. No. 7,425,436 (herein incorporated by reference in its entirety).

As used herein, the terms “capture ligand,” “capture moiety,” or “capture element refers to a ligand, substrate, etc., that forms a covalent bond with a capture protein upon interaction therewith. An example of a suitable capture ligand for use in embodiments of the present invention is the HALOTAG ligand described, for example, in U.S. Pat. No. 7,425,436 (herein incorporated by reference in its entirety). Moieties that find use as HALOTAG ligands include haloalkane (HA) groups (e.g., chloroalkane (CA) groups). In embodiments described herein that specify an HA or CA capture ligand, other suitable capture ligands may be substituted unless otherwise specified.

As used herein, the term “antibody” refers to a whole antibody molecule or a fragment thereof (e.g., fragments such as Fab, Fab′, and F(ab′)₂, variable light chain, variable heavy chain, Fv). It may be a polyclonal or monoclonal or recombinant antibody, a chimeric antibody, a humanized antibody, a human antibody, etc. As used herein, when an antibody or other entity “specifically recognizes” or “specifically binds” an antigen or epitope, it preferentially recognizes the antigen in a complex mixture of proteins and/or macromolecules and binds the antigen or epitope with affinity which is substantially higher than to other entities not displaying the antigen or epitope. In this regard, “affinity which is substantially higher” means affinity that is high enough to enable detection of an antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10¹ M⁻¹, >10¹⁰ M⁻¹, >10¹¹ M⁻¹, >10¹² M⁻¹, >10¹³ M⁻¹, etc.). In certain such embodiments, an antibody is capable of binding different antigens so long as the different antigens comprise that particular epitope. In certain instances, for example, homologous proteins from different species may comprise the same epitope.

As used herein, the term “antibody fragment” refers to a portion of a full-length antibody, including at least a portion of the antigen binding region or a variable region. Antibody fragments include, but are not limited to, Fab, Fab′, F(ab′)₂, Fv, scFv, Fd, variable light chain, variable heavy chain, diabodies, and other antibody fragments that retain at least a portion of the variable region of an intact antibody. See, e.g., Hudson et al. (2003) Nat. Med. 9:129-134; herein incorporated by reference in its entirety. In certain embodiments, antibody fragments are produced by enzymatic or chemical cleavage of intact antibodies (e.g., papain digestion and pepsin digestion of antibody) produced by recombinant DNA techniques, or chemical polypeptide synthesis. For example, a “Fab” fragment comprises one light chain and the C_{H1} and variable region of one heavy chain. The heavy chain of a Fab molecule cannot form a disulfide bond with another heavy chain molecule. A “Fab′” fragment comprises one light chain and one heavy chain that comprises an additional constant region extending between the C_{H1} and C_{H2} domains. An interchain disulfide bond can be formed between two heavy chains of a Fab′ fragment to form a “F(ab′)₂” molecule. An “Fv” fragment comprises the variable regions from both the heavy and light chains, but lacks the constant regions. A single-chain Fv (scFv) fragment comprises heavy and light chain variable regions connected by a flexible linker to form a single polypeptide chain with an antigen-binding region. Exemplary single chain antibodies are discussed in detail in WO 88/01649 and U.S. Pat. Nos. 4,946,778 and 5,260,203; herein incorporated by reference in their entireties. In certain instances, a single variable region (e.g., a heavy chain variable region or a light chain variable region) may have the ability to recognize and bind antigen. Other antibody fragments will be understood by skilled artisans.

As used herein, the terms “primary antibody,” “primary antibody fragment,” and “primary affinity molecule” generally refer to an antibody, antibody fragment, or affinity molecule that binds directly to a target (protein) of interest or a tag attached (e.g., covalently) thereto. As used herein, the terms “secondary antibody,” “secondary antibody fragment,” and “secondary affinity molecule” generally refer to an antibody, antibody fragment, or affinity molecule that binds directly to the primary antibody, antibody fragment, or affinity molecule. The secondary antibody may be conjugated to a detection label (e.g., component of a bioluminescent complex).

As used herein, the term “alkyl” means a straight or branched saturated hydrocarbon chain containing from 1 to 30 carbon atoms, for example 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈alkyl), 1 to 6 carbon atoms (C₁-C₆ alkyl), 1 to 4 carbon atoms (C₁-C₄ alkyl), 6 to 20 carbon atoms (C₆-C₂M alkyl), or 8 to 14 carbon atoms (C₈-C₁₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “haloalkyl” means an alkyl group, as defined herein, in which at least one hydrogen atom (e.g., one, two, three, four, five, six, seven or eight hydrogen atoms) is replaced by a halogen. The

### DETAILED DESCRIPTION

Provided herein are systems and methods for the detection and quantification of a target protein via bioluminescence and/or bioluminescent resonance energy transfer (BRET). In particular, provided herein are assay methods using a multi-component biochemical system that assembles into a detectable in vitro chemical complex at a protein target of interest.

Provided herein are in vitro target engagement assays in which binding of a reference ligand to a target protein results in the production of a detectable signal (e.g., luminescence, BRET, etc.) and the addition of an unlabeled test ligand displaces the reference ligand leading to a loss of the detectable signal that is proportional to the affinity of the test ligand for the target protein.

In a first aspect, provided herein are systems comprising (a) a first component of a bioluminescent complex tethered to a target protein; (b) a second component of the bioluminescent complex tethered to a reference ligand for the target protein; (c) a test ligand for the target protein; and (d) a substrate for the bioluminescent complex; wherein the bioluminescent complex emits a bioluminescent signal in the presence of the substrate; wherein when the reference ligand is bound to the target protein the first and second components of the bioluminescent complex are placed in proximity to allow formation of the active bioluminescent complex and produce a bioluminescent signal in the presence of the substrate for the bioluminescent complex; wherein if the test ligand is capable of binding to the target protein, the test ligand competes with the reference ligand for binding to the target protein, thereby decreasing the bioluminescent signal produced by the system. In some embodiments, methods are provided utilizing such a systems (e.g., to detect/quantify the binding of a test ligand to a target protein).

The first component of the bioluminescent complex and the target protein may be tethered together by any suitable arrangement, including but not limited to: (1) fusion (e.g., directly or via a linker sequence); (2) fusion of the first component of the bioluminescent complex to an antibody, antibody fragment, or other affinity molecule and linking of the target protein to a tag comprising an epitope for the antibody, antibody fragment, or other affinity molecule (or vice versa); (3) fusion of the first component of the bioluminescent complex to a modified dehalogenase capable of covalently binding to a haloalkyl ligand and linking of the target protein to a haloakyl group (or vice versa); (4) linking the first component of the bioluminescent complex to a secondary antibody, antibody fragment, or other affinity molecule capable of binding to a primary antibody, antibody fragment, or other affinity molecule capable of binding to the target protein or an affinity tag on the target protein; etc.

In a second aspect, provided herein are systems comprising (a) a bioluminescent protein tethered to a target protein; (b) a fluorophore tethered to a reference ligand for the target protein; (c) a test ligand for the target protein; and (d) a substrate for the bioluminescent protein; wherein the bioluminescent protein emits a bioluminescent signal at a first wavelength in the presence of the substrate; wherein the fluorophore absorbs light at the first wavelength and emits a fluorescent signal at the second wavelength; wherein when the reference ligand is bound to the target protein the fluorophore is in proximity to the bioluminescent protein to allow bioluminescence resonance energy transfer (BRET) from the bioluminescent protein; and wherein if the test ligand is capable of binding to the target protein the test ligand competes with the reference ligand for binding to the target protein, thereby decreasing the fluorescent signal resulting from the BRET. In some embodiments, methods are provided utilizing such a systems (e.g., to detect/quantify the binding of a test ligand to a target protein).

The bioluminescent protein and the target protein may be tethered together by any suitable arrangement, including but not limited to: (1) fusion (e.g., directly or via a linker sequence); (2) fusion of the bioluminescent protein to an antibody, antibody fragment, or other affinity molecule and linking of the target protein to a tag comprising an epitope for the antibody, antibody fragment, or other affinity molecule (or vice versa); (3) fusion of the bioluminescent protein to a modified dehalogenase capable of covalently binding to a haloalkyl ligand and linking of the target protein to a haloakyl group (or vice versa); (4) linking the bioluminescent protein to a secondary antibody, antibody fragment, or other affinity molecule capable of binding to a primary antibody, antibody fragment, or other affinity molecule capable of binding to the target protein or an affinity tag on the target protein; etc.

In a third aspect, provided herein are systems comprising (a) a first component of a bioluminescent complex tethered to a target protein; (b) a fluorophore tethered to a reference ligand for the target protein; (c) a second component of the bioluminescent complex having high affinity for the first component of a bioluminescent complex such that the bioluminescent complex forms between the first and second components without facilitation; (d) a test ligand for the target protein; and (e) a substrate for the bioluminescent complex; wherein the bioluminescent complex emits a bioluminescent signal at a first wavelength in the presence of the substrate; wherein the fluorophore absorbs light at the first wavelength and emits a fluorescent signal at the second wavelength; wherein when the reference ligand is bound to the target protein the fluorophore is in proximity to the bioluminescent complex to allow bioluminescence resonance energy transfer (BRET) from the bioluminescent complex; and wherein if the test ligand is capable of binding to the target protein the test ligand competes with the reference ligand for binding to the target protein, thereby decreasing the fluorescent signal resulting from the BRET. In some embodiments, methods are provided utilizing such a systems (e.g., to detect/quantify the binding of a test ligand to a target protein).

The first component of a bioluminescent complex and the target protein may be tethered together by any suitable arrangement, including but not limited to: (1) fusion (e.g., directly or via a linker sequence); (2) fusion of the first component of a bioluminescent complex to an antibody, antibody fragment, or other affinity molecule and linking of the target protein to a tag comprising an epitope for the antibody, antibody fragment, or other affinity molecule (or vice versa); (3) fusion of the first component of a bioluminescent complex to a modified dehalogenase capable of covalently binding to a haloalkyl ligand and linking of the target protein to a haloakyl group (or vice versa); (4) linking the first component of the bioluminescent complex to a secondary antibody, antibody fragment, or other affinity molecule capable of binding to a primary antibody, antibody fragment, or other affinity molecule capable of binding to the target protein or an affinity tag on the target protein; etc.

The present disclosure includes systems and methods comprising bioluminescent polypeptides, bioluminescent complexes, and components thereof. In particular, light emitted from bioluminescent proteins or complexes (or from luminophores acted upon by bioluminescent proteins or complexes) is used to detect/quantify a target protein and/or induce BRET detect/quantify a target protein in a biochemical assay.

In some embodiments, systems and methods herein comprise a bioluminescent protein. In some embodiments, a bioluminescent protein is a luciferase enzyme. Suitable luciferase enzymes include those selected from the group consisting of: Photinus pyralis or North American firefly luciferase; Luciola cruciata or Japanese firefly or genji-botaru luciferase; Luciola italic or Italian firefly luciferase; Luciola lateralis or Japanese firefly or Heike luciferase; N. nambi luciferase; Luciola mingrelica or East European firefly luciferase; Photuris pennsylvanica or Pennsylvania firefly luciferase; Pyrophorus plagiophthalamus or Click beetle luciferase; Phrixothrix hirtus or Railroad worm luciferase; Renilla reniformis or wild-type Renilla luciferase; Renilla reniformis Rluc8 mutant Renilla luciferase; Renilla reniformis Green Renilla luciferase; Gaussia princeps wild-type Gaussia luciferase; Gaussia princeps Gaussia-Dura luciferase; Cypridina noctiluca or Cypridina luciferase; Cypridina hilgendorfii or Cypridina or Vargula luciferase; Metridia longa or Metridia luciferase; TurboLuc (Auld et al. Biochemistry 2018, 57, 31, 4700-4706: incorporated by reference in its entirety); Nano-lanterns (Suzuki et al. Nature Communications volume 7, Article number: 13718 (2016); incorporated by reference in its entirety); and Oplophorus luciferase (e.g., Oplophorus gracilirostris (OgLuc luciferase), Oplophorus grimaldii, Oplophorus spinicauda, Oplophorus foliaceus, Oplophorus noraezeelandiae, Oplophorus typus, Oplophorus noraezelandiae or Oplophorus spinous).

In some embodiments, the bioluminescent protein is a luciferase of Oplophorus gracilirostris, the NANOLUC® luciferase (Promega Corporation; U.S. Pat. Nos. 8,557,970; 8,669,103; herein incorporated by reference in their entireties). PCT Appln. No. PCT/US2010/033449, U.S. Pat. No. 8,557,970, PCT Appln. No. PCT/2011/059018, and U.S. Pat. No. 8,669,103 (each of which is herein incorporated by reference in their entirety and for all purposes) describe compositions and methods comprising bioluminescent polypeptides. Such polypeptides find use in embodiments herein and can be used in conjunction with the compositions, assays, devices, systems, and methods described herein. In some embodiments, compositions, assays, devices, systems, and methods provided herein comprise a bioluminescent polypeptide of SEQ ID NO: 1, or having at least 60% (e.g., 60%, 65%, 70%, 75%, 80%, 85%, 90%, 95%, 96%, 97%, 98%, 99%, 100%, or ranges therebetween) sequence identity with SEQ ID NO: 1. In some embodiments, any of the aforementioned bioluminescent proteins are linked (e.g., fused, chemically linked, etc.) to one or more other components of the assays and systems described herein (e.g., fused to a HALOTAG protein, a target protein, an antibody or antibody fragment, etc.).

In some embodiments, a bioluminescent protein is a circularly permuted version of a natural or modified bioluminescent protein (See, e.g., U.S. Pat. No. 10,774,364; incorporated by reference in its entirety).

The native Oplophorus gracilirostris luciferase (OgLuc) and commercially-available NANOLUC luciferase (Promega Corporation) each comprise polypeptides of 10 β (beta) strands (β1, β2, β3, β4, β5, β6, β7, β8, β9, β10). U.S. Pat. No. 9,797,889 (herein incorporated by reference in its entirety) describes development and use of a complementation system comprising a β1-9-like polypeptide and a β10-like peptide (certain OgLuc/NANOLUC-based polypeptide and peptide sequences in polypeptide and peptide sequences in U.S. Pat. No. 9,797,889 differ from the corresponding sequences in NANOLUC and wild-type native OgLuc). Similarly, U.S. application Ser. No. 16/439,565 (herein incorporated by reference in its entirety) describes the development and use of a complementation systems comprising two or more OgLuc/NANOLUC peptides and/or polypeptides (certain OgLuc/NANOLUC-based polypeptide and peptide sequences in U.S. patent Ser. No. 16/439,565 differ from the corresponding sequences in NANOLUC and wild-type native OgLuc).

In some embodiments, systems and methods herein comprise a bioluminescent complex (e.g., two or more components (e.g., peptides and/or polypeptides) that combine through structural complementation to form a complex that is capable of activating a luminophore to emit light). In some embodiments, a luminophore emits significantly more light in the presence of the bioluminescent complex than in the presence of any one of the components alone). In some embodiments, a bioluminescent complex is formed from fragments (e.g., peptide(s) and/or polypeptide(s)) of a luciferase enzyme. In some embodiments, a bioluminescent complex is a circularly permuted version of a natural or modified bioluminescent component (e.g., formed from two fragments of a circularly permuted luciferase); See, e.g., U.S. Pat. No. 10,774,364; incorporated by reference in its entirety.

PCT Appln. Nos. PCT/US14/26354, PCT/US19/036844, and PCT/US20/62499; U.S. Pat. No. 9,797,889; U.S. patent application Ser. No. 16/439,565; and U.S. Pub. No. 2021/0262941 (each of which is herein incorporated by reference in their entirety and for all purposes) describe compositions and methods for the assembly of bioluminescent complexes; such complexes, and the peptide and polypeptide components thereof, find use in embodiments herein and can be used in conjunction with the assays and methods described herein.

In some embodiments, peptide and polypeptide components are provided for the assembly of a bioluminescent complex, capable of generating luminescence in the presence of an appropriate substrate (e.g., a coelenterazine or a coelenterazine analog (e.g., furimazine, fluorofurimazine, etc.). In some embodiments, complementary polypeptide(s) and peptide(s) collectively span the length (or >75% of the length, >80% of the length, >85% of the length, >90% of the length, >95% of the length, or more) of a luciferase base sequence (or collectively comprise at least 40% sequence identity to a luciferase base sequence (e.g., >40%, >45%, >50%, >55%, >60%, >65%, >70%, >75% >80%, >85%, >90%, >95%, or more). In some embodiments, “complementary” polypeptide(s) and peptide(s) are separate molecules that each correspond to a portion of a luciferase base sequence. Through structural complementarity, they assemble to form a bioluminescent complex. Suitable luciferase base sequences may include SEQ ID NOS: 1 or 2, or the sequences of any of the full-length luciferases listed above. In some embodiments, the bioluminescent complex comprises the NANOBIT or NANOTRIP systems (Promega; Madison, WI). In some embodiments, the peptide and/or polypeptide components of a bioluminescent complex collectively comprise at least 60% sequence identity (e.g., >60%, >65%, >70%, >75%, >80%, >85%, >90%, >95%, >99%) with SEQ ID NO: 1 and/or SEQ ID NO: 2. In some embodiments, the peptide and/or polypeptide components of the bioluminescent complex comprise HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7). In some embodiments, the peptide and/or polypeptide components of the bioluminescent complex comprise at least 60% sequence identity (e.g., >60%, >65%, >70%, >75%, >80%, >85%, >90%, >95%, >99%) with HIBIT (SEQ ID NO: 3), SMBIT (SEQ ID NO: 4), LGBIT (SEQ ID NO: 5), LGTRIP (SEQ ID NO: 6), and/or SMTRIP9 (SEQ ID NO: 7).

In some embodiments, any of the aforementioned components of bioluminescent complexes are linked (e.g., fused, chemically linked, linked via primary/secondary antibodies, tethered, etc.) to one or more other components of the assays and systems described herein (e.g., fused to a HALOTAG protein, an antibody, etc.).

There are various characteristics of the bioluminescent complexes that find use in embodiments herein that may provide advantages in certain applications. For example, a bioluminescent complex (e.g., a complex formed upon complementation of HIBIT/LGBIT) only generates light upon complementation of its component peptide/polypeptides; therefore, directly or indirectly conjugating (e.g., fusing, tethering, etc.) one or more components of the bioluminescent complex to other components of the system (e.g., photocatalyst, activatable molecule, target, etc.) ensuring the proximity of that component to the bioluminescent complex upon light generation. Tethering of two other components of the system to separate components of the bioluminescent complex ensures the proximity of those components upon light generation by the complex. In some embodiments, the use of a bioluminescent complex, due to the requirement that two components come together to form the complex, provides enhanced spatiotemporal resolution through conditional activation at a specific site.

In some embodiments the bioluminescent protein or a component of the multipart bioluminescent complex is inserted in an internal position within the capture agent. In some embodiments, a position within the capture agent is selected to increase efficiency of bioluminescence activation of the catalyst through greater proximity or favorable conformation.

In some embodiments, the bioluminescent protein or a component of the multipart bioluminescent complex is circularly permuted.

### Luminophore Substrate

In some embodiments, the systems and methods herein comprise luminophore substrates that emit light upon interaction with the bioluminescent proteins and/or complexes described herein. Suitable luminophores for the bioluminescent protein or complex used in the system or method will be understood. For example, firefly luciferin, with the structure:

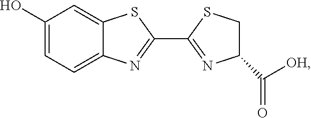 [SMILES: O=C(O)[C@H]1CSC(c2nc3ccc(O)cc3s2)=N1]

is the luciferin found in many Lampyridae species, and is the substrate of beetle luciferases.

Latia luciferin, with the structure:

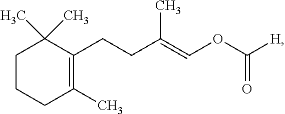

is from the freshwater snail Latia neritoides.

Bacterial luciferin, with the structure:

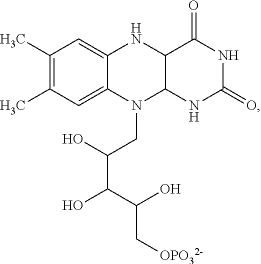

finds use as a substrate for many bacterial luciferases.

Coelenterazine of the structure:

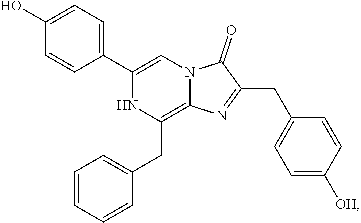 [SMILES: O=c1c(Cc2ccc(O)cc2)nc2c(Cc3ccccc3)[nH]c(-c3ccc(O)cc3)cn1-2]

is found in radiolarians, ctenophores, cnidarians, squid, brittle stars, copepods, chaetognaths, fish, and shrimp, and is the luminophore substrate for the luciferases of those organisms. Variants and derivatives of coelenterazine, such as furimazine and fluorofurimazine find use in embodiments herein (e.g., with Oplophorus-derived bioluminescent proteins and complexes).

Other luminophore substrates include those of dinoflagellates:

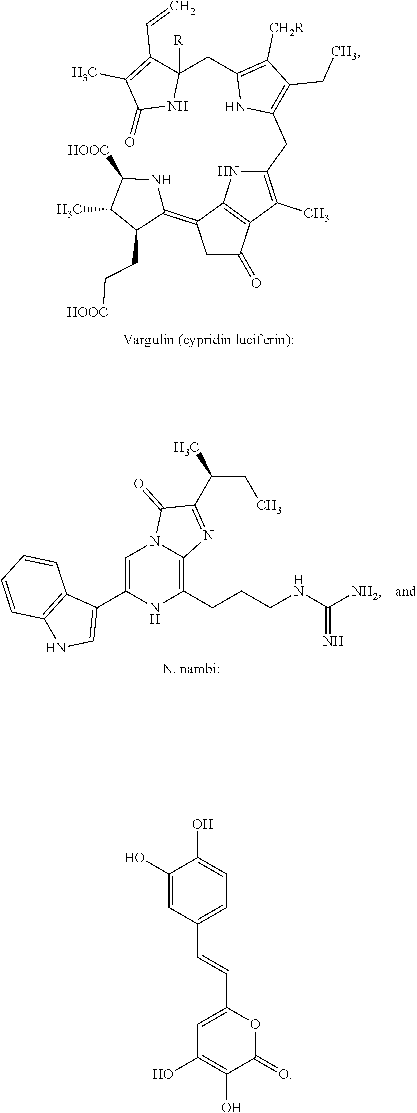

Pairing of appropriate bioluminescent proteins or complexes with luminophores is understood in the field. In particular embodiments, a bioluminescent protein is provided in a system or method herein that utilizes an imidazopyrazine luminophore, such as coelenterazine, furimazine, or fluorofurimazine (U.S. application Ser. No. 16/548,214; incorporated by reference in its entirety). In some embodiments, a system or method comprises (1) an Oplophorus-derived polypeptide (e.g., NANOLUC) or components of an Oplophorus-derived bioluminescent complex (e.g., NANOBIT, NANOTRIP) and an imidazopyrazine luminophore (e.g., coelenterazine, furimazine, fluorofurimazine, etc.). In some embodiments, systems and methods herein comprise an imidazopyrazine luminophore such as native coelenterazine, furimazine, fluorofurimazine, coelenterazine-n, coelenterazine-f, coelenterazine-h, coelenterazine-hcp, coelenterazine-cp, coelenterazine-c, coelenterazine-e, coelenterazine-fcp, bis-deoxycoelenterazine (“coelenterazine-hh”), coelenterazine-i, coelenterazine-icp, coelenterazine-v, and 2-methyl coelenterazine, in addition to those disclosed in WO 2003/040100; U.S. application Ser. No. 12/056,073 (paragraph [0086]); and U.S. Pat. No. 8,669,103; the disclosures of which are incorporated by reference herein in their entireties.

In some embodiments, the luminophore emits light upon interaction with the bioluminescent protein or complex. In some embodiments, the luminophore emits light in the visible light spectrum (e.g., about 400 to about 700 nm (e.g., 400 nm, 425 nm, 450 nm, 475 nm, 500 nm, 525 nm, 550 nm, 575 nm, 600 nm, 625 nm, 650 nm, 675 nm, 700 nm, or ranges therebetween). In some embodiments, the luminophore emits light of a wavelength between 400 and 500 nm (e.g., 400 nm, 410 nm, 420 nm, 430 nm, 440 nm, 450 nm, 460 nm, 470 nm, 480 nm, 490 nm, 500 nm, or ranges therebetween).

### Fluorophores

Some embodiments herein utilize a fluorophore (e.g., small molecule fluorophore or fluorescent protein) to produce a detectable signal. In particular embodiments, the fluorophore serves as a BRET acceptor in the systems and methods herein. Therefore, any fluorophore may find use in embodiments herein, provided it is paired with a bioluminescent protein or bioluminescent complex with an emission spectrum capable of exciting the fluorophore.

In some embodiments, the fluorophore is a small molecule (e.g., molecular weight less than 3,000 daltons, <2,500 daltons, <2,000 daltons, <1,500 daltons, <1,000 daltons, <900 daltons, <800 daltons, <700 daltons, <600 daltons).

Suitable fluorophores for use as fluorescent moieties herein include, but are not limited to: stilbazolium derivatives (Marquesa et al. Mechanism-Based Strategy for Optimizing HALOTAG Protein Labeling. ChemRxiv. Cambridge: Cambridge Open Engage; 2021; incorporated by reference in its entirety), xanthene derivatives (e.g., fluoresceins, rhodamines, rhodols, Oregon green, eosin, Texas red, etc.), cyanine derivatives (e.g., cyanine, indocarbocyanine, oxacarbocyanine, thiacarbocyanine, merocyanine, etc.), naphthalene derivatives (e.g., dansyl and prodan derivatives), oxadiazole derivatives (e.g., pyridyloxazole, nitrobenzoxadiazole, benzoxadiazole, etc.), pyrene derivatives (e.g., cascade blue), oxazine derivatives (e.g., Nile red, Nile blue, cresyl violet, oxazine 170, etc.), acridine derivatives (e.g., proflavin, acridine orange, acridine yellow, etc.), arylmethine derivatives (e.g., auramine, crystal violet, malachite green, etc.), CF dye (Biotium), BODIPY (Invitrogen), ALEXA FLUOR (Invitrogen), DYLIGHT FLUOR (Thermo Scientific, Pierce), ATTO and TRACY (Sigma Aldrich), FluoProbes (Interchim), DY and MEGASTOKES (Dyomics), SULFO CY dyes (CYANDYE, LLC), SETAU AND SQUARE DYES (SETA BioMedicals), QUASAR and CAL FLUOR dyes (Biosearch Technologies), SURELIGHT DYES (APC, RPE, PerCP, Phycobilisomes)(Columbia Biosciences), etc.

In some embodiments, the fluorophore is a rhodol or rhodamine dye (Beija et al. Chem. Soc. Rev., 2009, 38, 2410-2433; incorporated by reference in its entirety) or a variant or derivative thereof. In some embodiments, the rhodol or rhodamine dye is selected from:

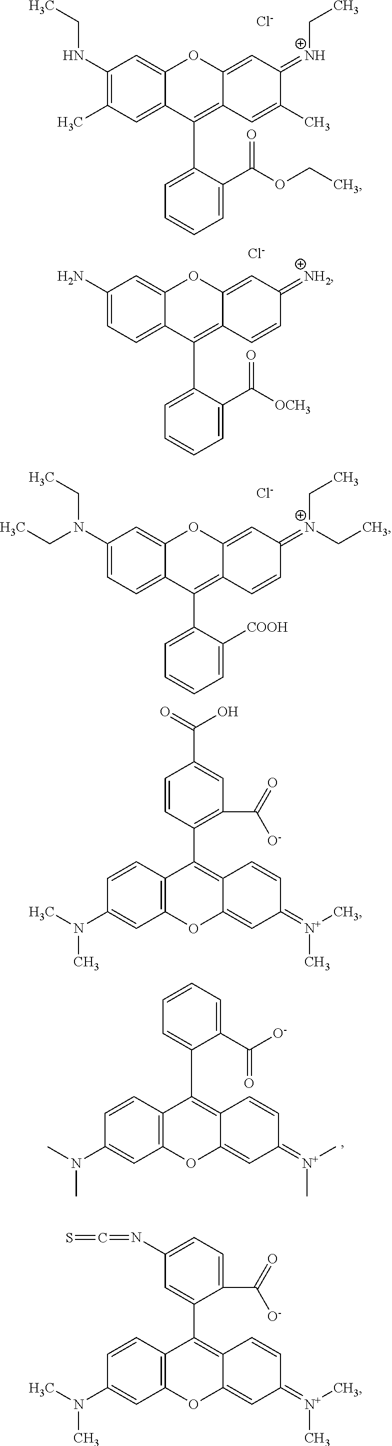

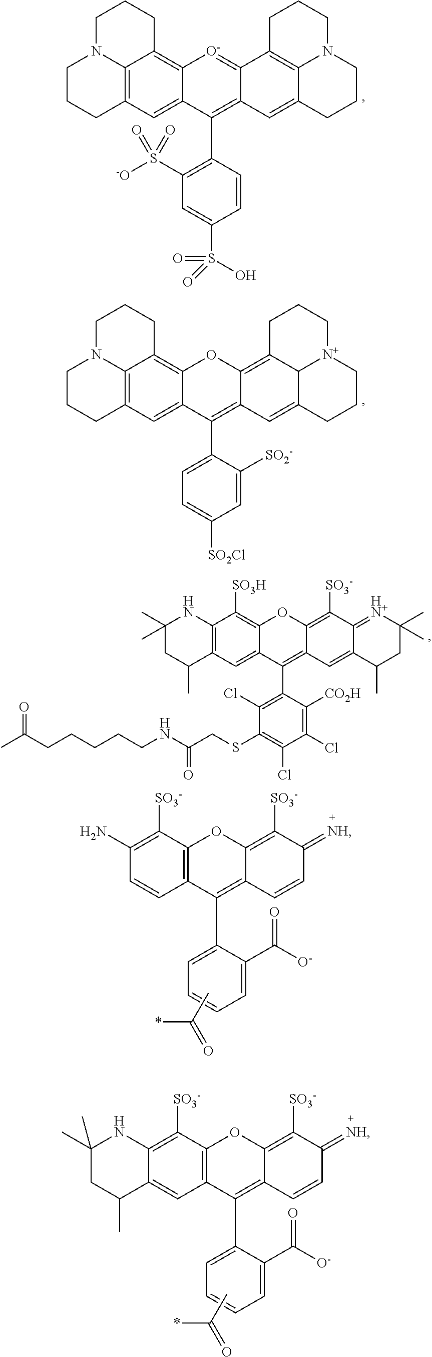

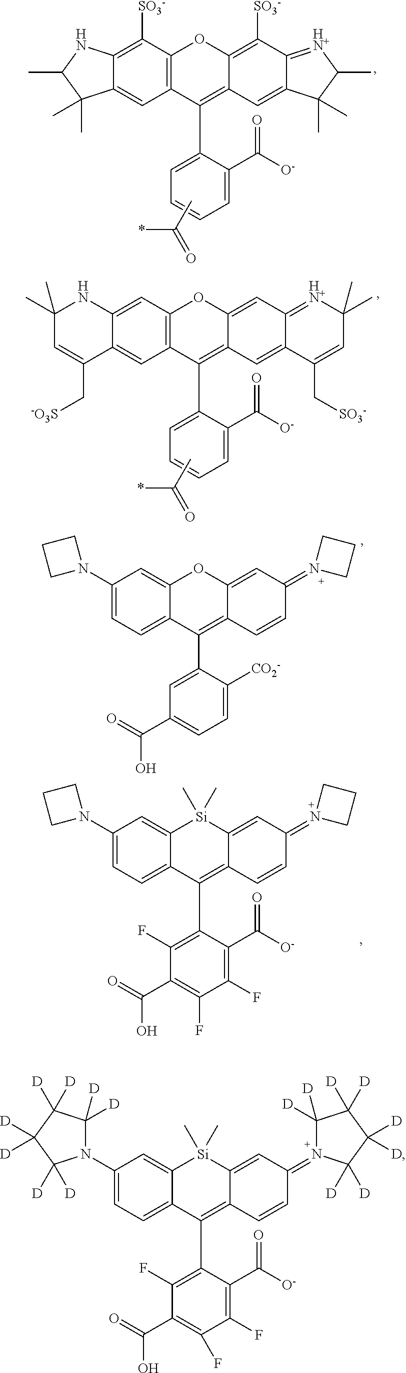

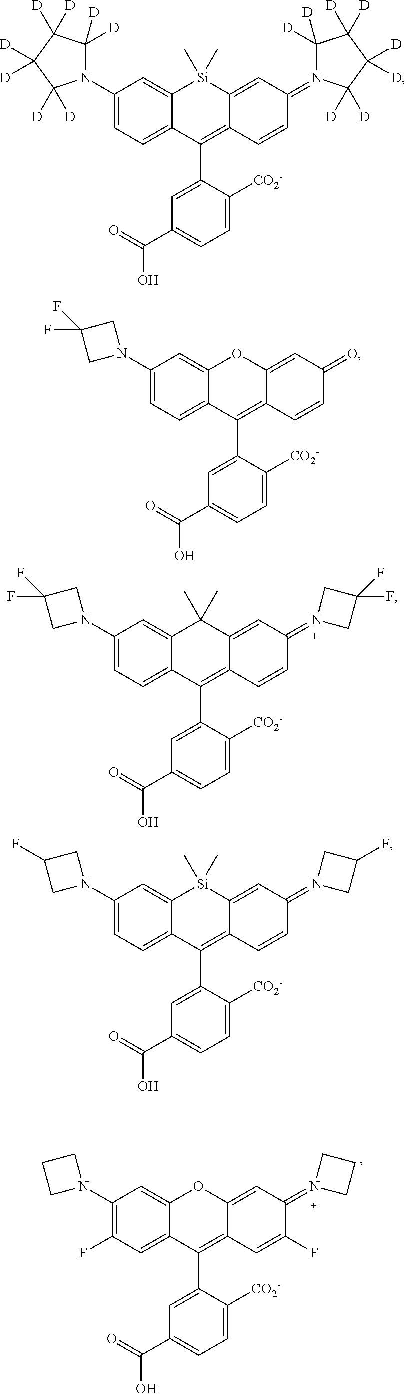

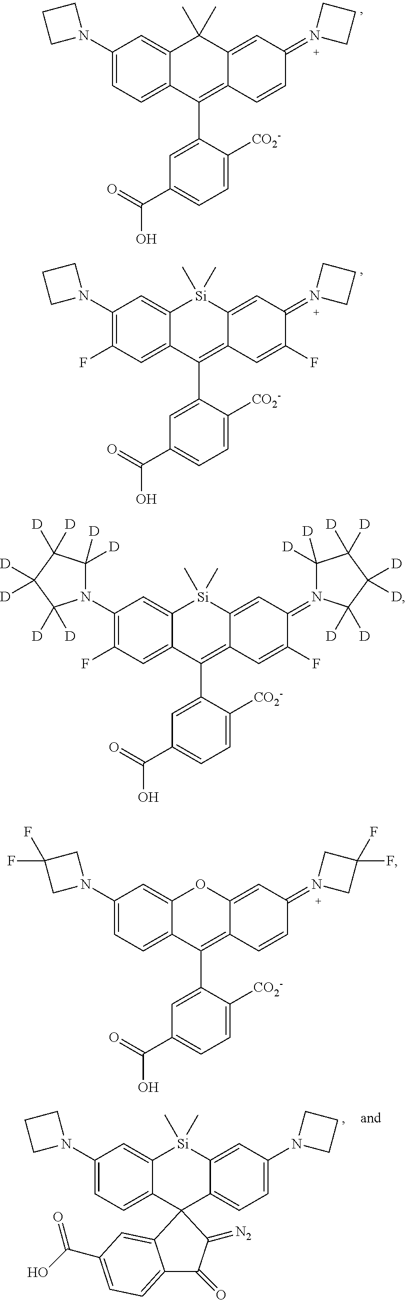

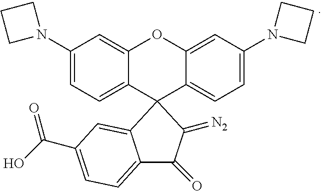

In some embodiments, the fluorophore is a rhodamine.

In some embodiments, the fluorescent protein is selected from yellow fluorescent protein (YFP), green fluorescent protein (GFP), cyan fluorescent protein (CFP), red fluorescent protein (RFP), umbelliferone, fluorescein, fluorescein isothiocyanate, rhodamine, dichlorotriazinylamine fluorescein, cyanines, dansyl chloride, phycocyanin, and phycoerythrin.

### HALOTAG

In some embodiments, provided herein are compositions (e.g., fusion peptides and polypeptides) and systems (e.g., multiple complementary fusion peptides and polypeptides, substrates, ligands, etc.) comprising complementary peptide/polypeptide fragments capable of interacting (e.g., facilitated or unfacilitated) to form an active modified dehalogenase complex capable of forming a covalent bond to a haloalkane ligand. In some embodiments, a first fusion is provided comprising a complementary peptide fragment of a modified dehalogenase, and a second fusion is provided comprising a complementary polypeptide fragment of the modified dehalogenase, wherein upon interacting (e.g., facilitated or unfacilitated), the complementary peptide and polypeptide form an active modified dehalogenase complex capable of forming a covalent bond to a haloalkane ligand. In some embodiments, a first fusion is provided comprising a complementary peptide fragment of a modified dehalogenase, and a complementary polypeptide fragment of the modified dehalogenase is provided (e.g., not as a fusion), wherein upon interacting (e.g., facilitated or unfacilitated), the complementary peptide and polypeptide form an active modified dehalogenase complex capable of forming a covalent bond to a haloalkane ligand. In some embodiments, the complementary peptide and polypeptide are fragments of a split mutant dehalogenase. In alternative embodiments, both fragments may be polypeptides.

Provided herein, as components of the compositions, systems, and methods herein are split mutated dehalogenases, such as those derived from the commercially available HALOTAG protein (Promega) and/or mutated dehalogenases disclosed in U.S. published application 20060024808, the disclosure of which is incorporated by reference herein.

Even though these mutant dehalogenases are not technically enzymes (no substrate turnover), the stable binding of a substrate thereto is dependent on proper protein structure. The consequence of re-associating the split fragments of a mutant dehalogenase differs from that of a split enzyme system because the labeling function of a mutant dehalogenase is retained on one of the fragments even after it has separated from its partner, whereas split enzymes are only active when they are brought together and bear no artifact of their prior activity after they are separated. In effect, the labeling reaction of a split mutant dehalogenase provides a molecular memory of a protein interaction. In the case of fluorogenic ligands, the label is retained on one of the fragments, but may not be detectable after complex dissociation (since the fluorogen-activating contacts with the protein may be disrupted/absent); therefore, the combination of split dehalogenase and fluorogenic ligands produce a unique situation of permanent labeling, but with dynamic (on/off) fluorescence detection of the retained label.

A mutant dehalogenase provides for efficient labeling within a living cell or lysate thereof. This labeling is only conditional on the presence or expression of the protein and the presence of the labeled hydrolase substrate. In contrast, the labeling of a split mutant dehalogenase is dependent on a specific protein interaction occurring within the cell and the presence of the labeled hydrolase substrate.

In some embodiments, provided as a component of the compositions, systems, and methods herein are split modified dehalogenases. In some embodiments, a first fragment of a mutant dehalogenase is fused to a first fragment of luminescent protein (e.g., and optionally a protein or molecule of interest), and a second fragment of the mutant dehalogenase is fused to a second fragment of the luminescent protein (e.g., and optionally a protein or molecule of interest)). In some embodiments, at least one of the mutant dehalogenase fragments has a substitution that if present in a full-length modified dehalogenase having the sequence of the two fragments, forms a bond with a haloalkane ligand. In some embodiments, the first fragment of the mutant dehalogenase and the second fragment of the mutant dehalogenase are capable of interacting (e.g., facilitated or unfacilitated) to form an active modified dehalogenase complex.

HALOTAG is a 297-residue self-labeling polypeptide (33 kDa) derived from a bacterial hydrolase (dehalogenase) enzyme, which has been modified to covalently bind to its ligand, a haloalkane moiety. The HALOTAG ligand can be linked to solid surfaces (e.g., beads) or functional groups (e.g., fluorophores), and the HALOTAG polypeptide can be fused to various proteins of interest, allowing covalent attachment of the protein of interest to the solid surface or functional group.

The HALOTAG polypeptide is a modified dehalogenase with a genetically modified active site, which specifically binds to the haloalkane ligand chloroalkane linker with an enhanced and increased rate of ligand binding (Pries et al. The Journal of Biological Chemistry. 270(18):10405-11; incorporated by reference in its entirety). The reaction that forms the bond between the protein tag and chloroalkane linker is fast and essentially irreversible under physiological conditions (Waugh D S (June 2005). Trends in Biotechnology. 23(6):316-20; incorporated by reference in its entirety). In the natural hydrolase enzyme, nucleophilic attack of the chloroalkane reactive linker causes displacement of the halogen with an amino acid residue, which results in the formation of a covalent alkyl-enzyme intermediate. This intermediate would then be hydrolyzed by an amino acid residue within the wild-type hydrolase (Chen et al. (February 2005) Current Opinion in Biotechnology. 16(1):35-40; incorporated by reference in its entirety). This would lead to regeneration of the enzyme following the reaction. However, with HALOTAG, the modified haloalkane dehalogenase, the reaction intermediate cannot proceed through the second reaction because it cannot be hydrolyzed due to the mutation in the enzyme. This causes the intermediate to persist as a stable covalent adduct with which there is no associated back reaction (Marks et al. (August 2006) Nature Methods. 3 (8): 591-6; incorporated by reference in its entirety).

HALOTAG fusion proteins can be expressed using standard recombinant protein expression techniques (Adams et al. (May 2002) Journal of the American Chemical Society. 124(21):6063-76; incorporated by reference in its entirety). Since the HALOTAG polypeptide is a relatively small protein, and the reactions are foreign to mammalian cells, there is no interference by endogenous mammalian metabolic reactions (Naested et al. The Plant Journal. 18(5):571-6; incorporated by reference in its entirety). Once the fusion protein has been expressed, there is a wide range of potential areas of experimentation including enzymatic assays, cellular imaging, protein arrays, determination of sub-cellular localization, and many additional possibilities (Janssen D B (April 2004). Current Opinion in Chemical Biology. 8(2):150-9; incorporated by reference in its entirety).

In some embodiments, a ligand for a modified dehalogenase is provided herein. In some embodiments, the ligand is of the structure R-linker-A-X, wherein R the target protein (protein of interest), wherein A is (CH₂)₂₋₁₂, wherein X is a halogen, and wherein the linker is a linker moiety capable of tethering R to A-X. In some embodiments, the linker is a multiatom straight or branched chain including C, N, S, or O, or a group that comprises one or more rings, e.g., saturated or unsaturated rings, such as one or more aryl rings, heteroaryl rings, or any combination thereof. In some embodiments, the linker comprises a combination of —O(CH₂)₂— —(CH2)O—, —CH₂—, —NHC(O)O—, —OC(O)NH—, NHC(O)—, and —C(O)NH—. In some embodiments, the linker is 5 to 50 (e.g., 5, 10, 15, 20, 25, 30, 35, 40, 45, 50, or ranges therebetween) atoms in length. In some embodiments, the length of the linker for tethering the fluorophore allows for optimization of proximity and geometry (e.g., for efficient energy transfer).

Various HALOTAG ligands, functional groups, fusions, assays, modifications, uses, etc. are described in U.S. Pat. Nos. 8,748,148; 9,593,316; 10,246,690; 8,742,086; 9,873,866; 10,604,745; U.S. Pat. App. 2009/0253131; U.S. Pat. App. 2010/0273186; 20130337539; U.S. Pat. App. 2012/0258470; U.S. Pat. App. 2012/0252048; U.S. Pat. App. 2011/0201024; U.S. 2014/0322794; each of which is incorporated by reference in their entireties.

### Tethering

The systems and methods herein comprise various assay components tethered together to produce detectable signal that is dependent upon binding of a reference ligand to a target protein.

In some embodiments, a reference ligand is tethered to a component of a bioluminescent complex (e.g., SmBiT).

In some embodiments, a reference ligand is tethered to a fluorophore. The reference ligand may be tethered to the ligand directly or via a chemical linker.

A linker tethering a reference ligand to a fluorophore may include various combinations of such groups to provide linkers having ester (—C(O)O—), amide (—C(O)NH—), carbamate (—NHC(O)O—), urea (—NHC(O)NH—), phenylene (e.g., 1,4-phenylene), straight or branched chain alkylene, and/or oligo- and poly-ethylene glycol (—(CH₂CH₂O)ₓ—) linkages, and the like. In some embodiments, the linker may include 2 or more atoms (e.g., 2-200 atoms, such as 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, or 200 atoms, or any range therebetween (e.g., 2-20, 5-10, 15-35, 25-100, etc.)). In some embodiments, the linker includes a combination of oligoethylene glycol linkages and carbamate linkages. In some embodiments, the linker has a formula —O(CH₂CH₂O)_{z1}—C(O)NH—(CH₂CH₂O)_{z2}—C(O)NH—(CH₂)_{z3}—(OCH₂CH₂)_{z4}O—, wherein z1, z2, z3, and z4 are each independently selected form 0, 1, 2, 3, 4, 5, and 6. For example, in some embodiments, the linker has a formula selected from:

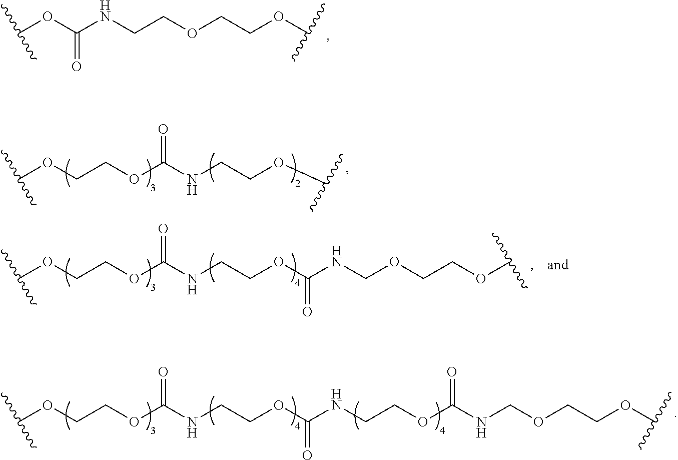

Non-limiting examples of compounds comprising the above fluorophores (R) include:

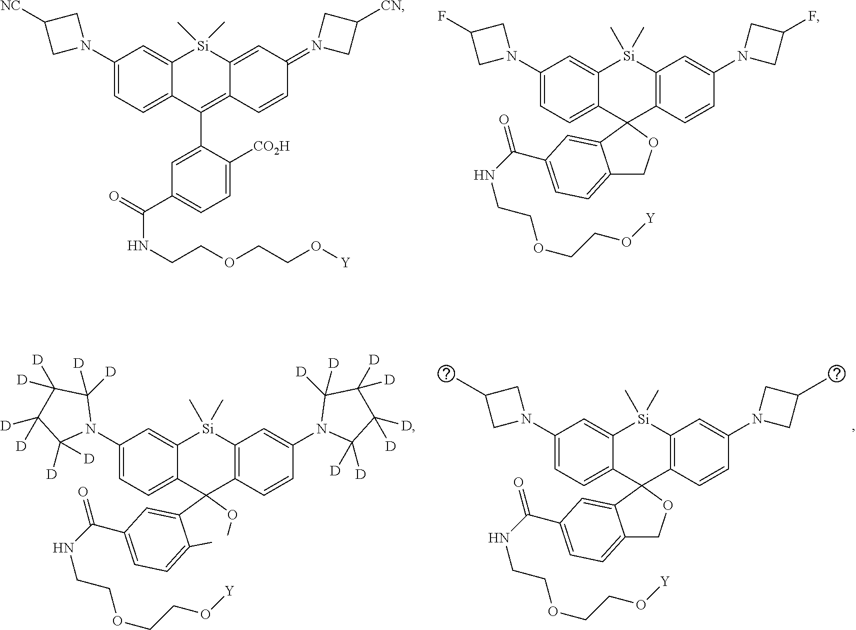

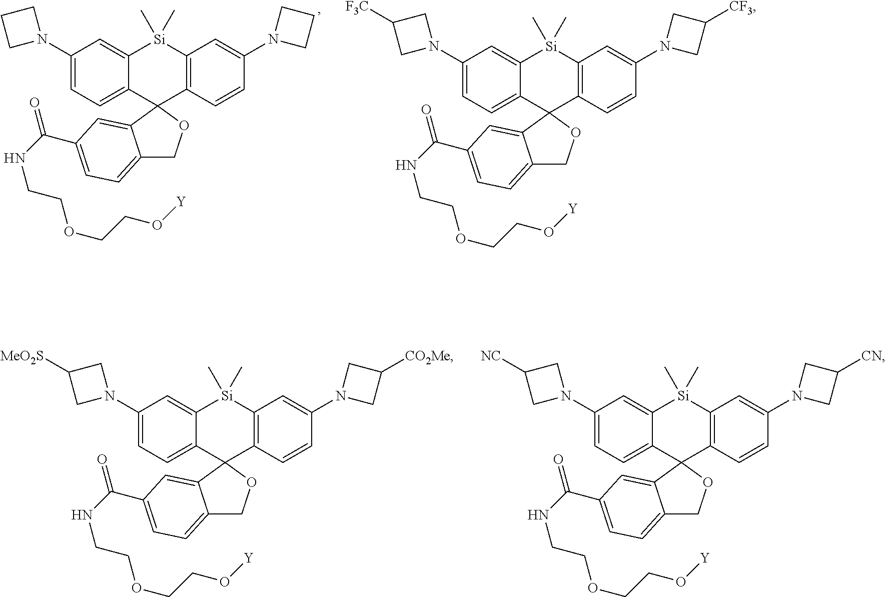

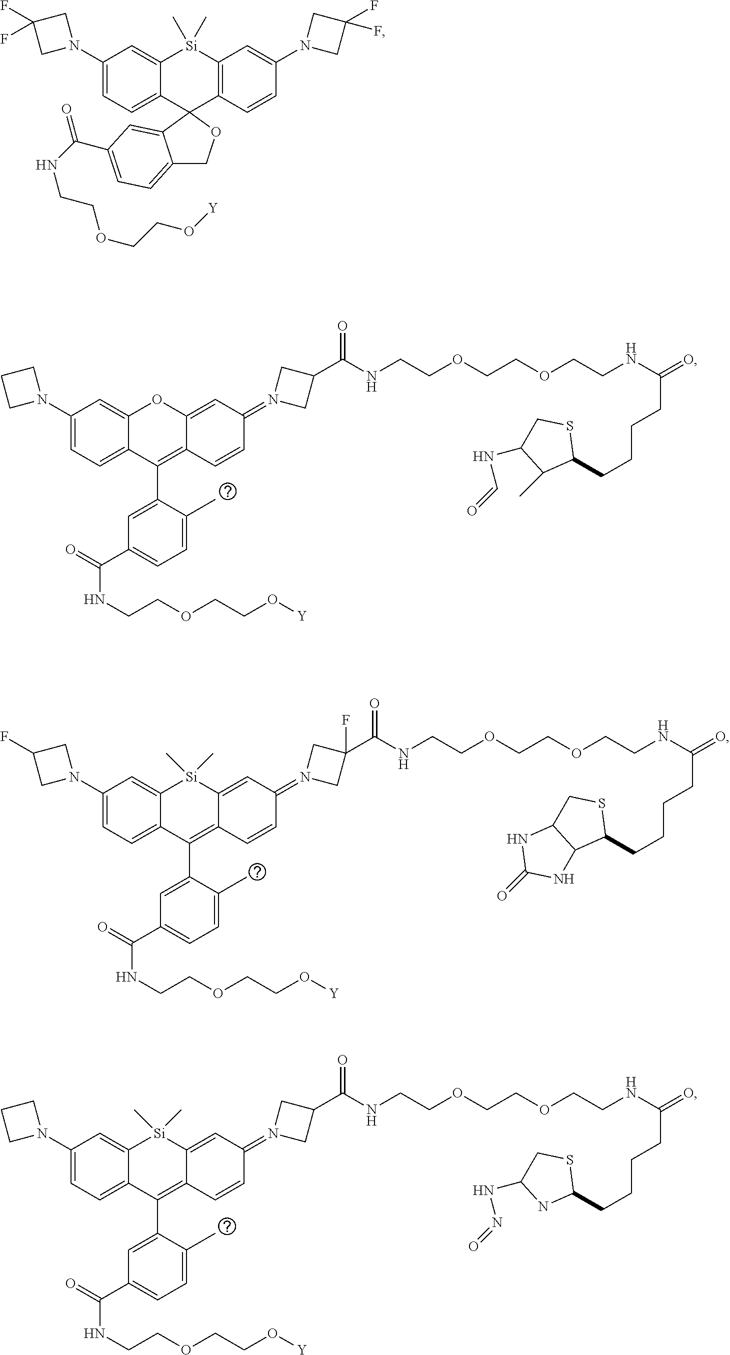

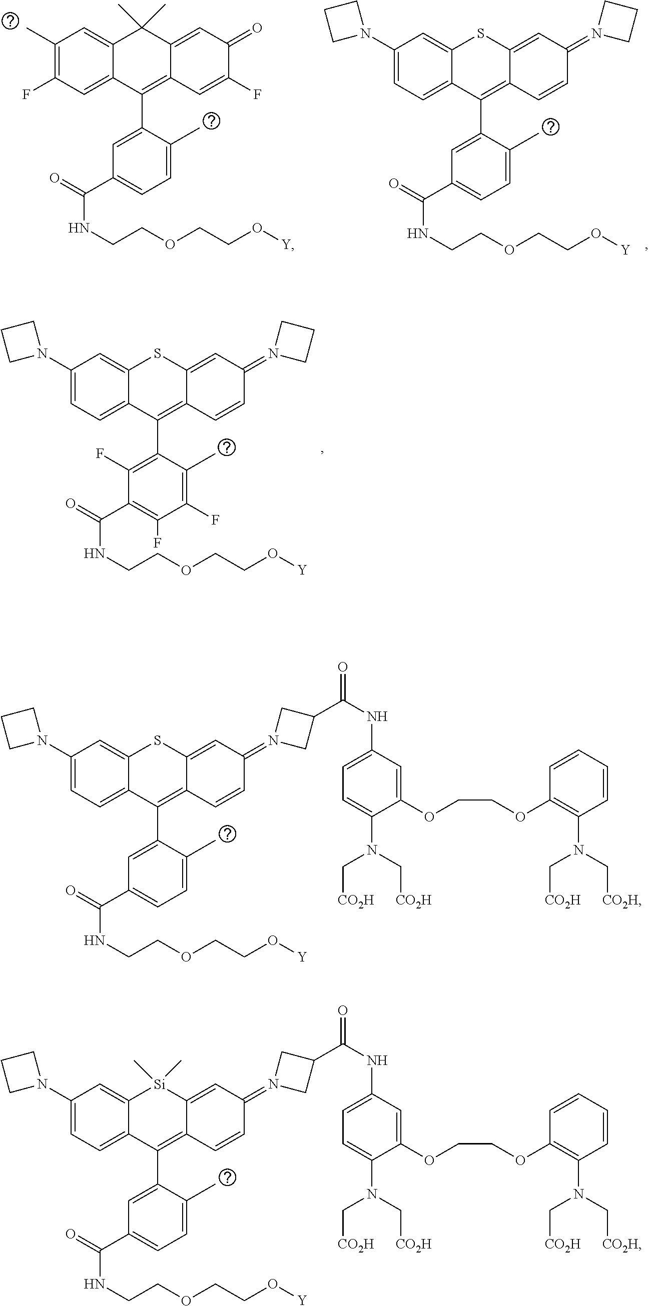

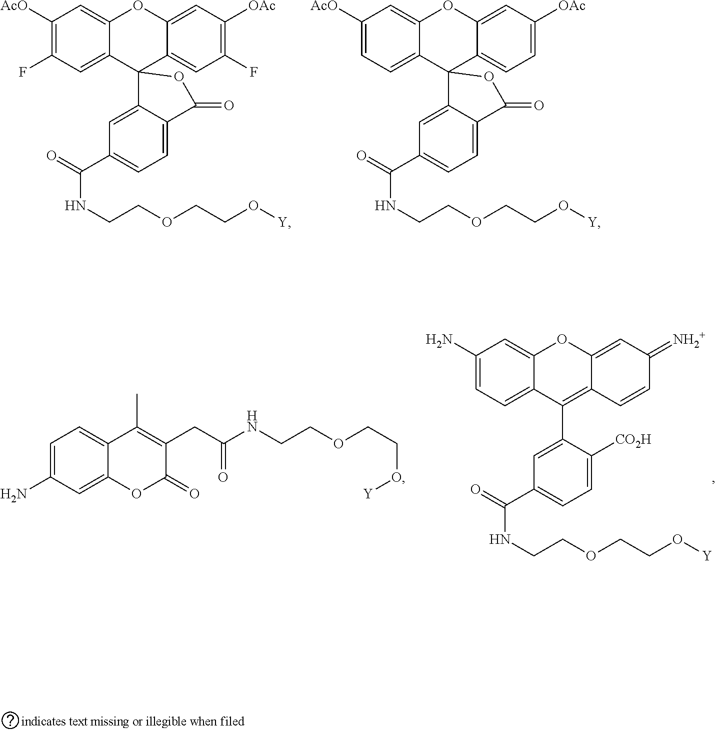

wherein Y is a
reference ligand. Although the exemplary compounds above have linkers comprising

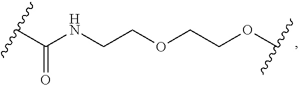 [SMILES: *OCCOCCNC(*)=O]

reference ligands may be tethered to fluorophores using any suitable chemical linker, including but not limited to those described above.

Embodiments herein comprise a protein of interest (e.g., target protein) tethered to a bioluminescent protein (e.g., NANOLUC) or component of a bioluminescent complex (e.g., HIBIT, LGBIT, etc.) by any suitable means of tethering two peptide/polypeptide components.

In some embodiments, systems and methods herein comprise a fusion of a protein of interest (e.g., target protein) and a bioluminescent protein (e.g., NANOLUC) or component of a bioluminescent complex (e.g., HIBIT, LGBIT, etc.). The POI may be directly fused to the bioluminescent protein or component of a bioluminescent complex or the fusion may comprise a linker peptide/polypeptide sequence. The linker may be 1-100 amino acids in length (e.g., 1, 2, 3, 4, 5, 10, 15, 20, 25, 30, 40, 50 60, 70, 80, 90, 100, or ranges therebetween). The linker may be of any suitable sequence that allows the tethered components to properly function and to be properly aligned for the assay.

In some embodiments, two components of a system herein are tethered together via the interaction of an affinity molecule (e.g., antibody, antibody fragment, streptavidin, etc.) and an affinity tag (e.g., epitope, biotin, etc.). In some embodiments, the affinity molecule (e.g., antibody, antibody fragment, streptavidin, etc.) is fused to one component (e.g., LGBIT, POI, etc.) and the affinity tag (e.g., epitope, biotin, etc.) is fused to the second component. Interaction of the affinity molecule and tag in a system herein results in tethering of the two components.

In some embodiments, two components of a system herein are tethered together via the interaction of a secondary affinity molecule (e.g., antibody, antibody fragment, etc.) with a primary affinity molecule (e.g., antibody, antibody fragment, streptavidin, etc.) that is capable of binding to a component of the system or an affinity tag (e.g., epitope, biotin, etc.) linked thereto. In some embodiments, a first component of the system (e.g., component of the bioluminescent complex, bioluminescent protein, etc.) becomes tethered to a second component of the system (e.g., target protein) when a secondary affinity molecule linked to the first component binds to a primary affinity molecule bound to the target protein (or an affinity tag thereon).

In some embodiments, two components of a system herein are tethered together via the interaction of an capture agent (e.g., HALOTAG) and an capture ligand (e.g., chloroalkane molecule). In some embodiments, the capture agent (e.g., HALOTAG) is fused to one component (e.g., LGBIT, POI, etc.) and the capture ligand (e.g., chloroalkane molecule) is chemically linked to the second component. Covalent binding of the capture agent and capture ligand in a system herein results in tethering of the two components.

In some embodiments, two components of a system herein are tethered together via a chemical linkage. Chemical linkage may be by any suitable conjugation method, such as, click chemistry, thiol-maleimide linkage, cysteine-maleimide-cysteine conjugation, etc. U.S. Pub No. 2020/0166460 (incorporated by reference in its entirety) describes peptides labeled with sulfo n-hydroxysuccinimidyl ester (sulfo-SE) moieties and the use of such reactive labeled peptides for the labeling of proteins via covalent conjugation to primary amines on the protein. Such chemistry can be employed herein to covalently link peptides (e.g., HiBiT) or small molecules (e.g., chloroalkane) to primary amines on proteins (e.g., POI, LgBiT, etc.) with the systems herein.

### Target Proteins and Reference Ligands

There are many proteins with known ligands that can serve as the target proteins and reference ligands in the systems here. A non-exhaustive list is provided in Table 1.

TABLE 1
Exemplary protein targets and reference ligands

| Protein Target | Ligands |
|---|---|
| Cyclooxygenase (COX) | Aspirin, Ibuprofen, Naproxen |
| HMG-CoA Reductase | Atorvastatin, Simvastatin, Lovastatin |
| Angiotensin-Converting Enzyme (ACE) | Lisinopril, Enalapril, Ramipril |
| Dipeptidyl Peptidase-4 (DPP-4) | Sitagliptin, Saxagliptin, Linagliptin |
| Beta-Adrenergic Receptors | Propranolol, Atenolol, Metoprolol |
| Histamine H1 Receptor | Diphenhydramine, Loratadine, Cetirizine |
| Serotonin 5-HT Receptors | Fluoxetine, Sertraline, Sumatriptan |
| Dopamine D2 Receptor | Haloperidol, Risperidone, Aripiprazole |
| Voltage-Gated Sodium Channels | Lidocaine, Phenytoin, Carbamazepine |
| Calcium Channels (L-type) | Verapamil, Diltiazem, Amlodipine |
| Potassium Channels (KATP) | Glibenclamide, Glipizide, Repaglinide |
| Sodium-Glucose Cotransporter 2 (SGLT2) | Dapagliflozin, Canagliflozin, Empagliflozin |
| Serotonin Transporter (SERT) | Citalopram, Escitalopram, Paroxetine |
| Estrogen Receptor | Tamoxifen, Raloxifene, Fulvestrant |
| Glucocorticoid Receptor | Prednisone, Dexamethasone, |
| | Hydrocortisone |
| Peroxisome Proliferator-Activated Receptor | Pioglitazone, Rosiglitazone, Fenofibrate |
| (PPAR) | |
| Mu-Opioid Receptor | Morphine, Fentanyl, Naloxone |
| Cannabinoid Receptors (CB1, CB2) | THC, Cannabidiol (CBD), Rimonabant |
| Epidermal Growth Factor Receptor (EGFR) | Gefitinib, Erlotinib, Cetuximab |
| Tumor Necrosis Factor (TNF) | Infliximab, Adalimumab, Etanercept |
| BCR-ABL Tyrosine Kinase | Imatinib, Dasatinib, Nilotinib |
| Vascular Endothelial Growth Factor (VEGF) | Bevacizumab, Ranibizumab, Aflibercept |
| Proteasome | Bortezomib, Carfilzomib, Ixazomib |
| Programmed Cell Death Protein 1 (PD-1) | Pembrolizumab, Nivolumab, Cemiplimab |
| Programmed Cell Death Ligand 1 (PD-L1) | Atezolizumab, Durvalumab, Avelumab |
| CD20 | Rituximab, Obinutuzumab, Ofatumumab |
| Interleukin-6 Receptor (IL-6R) | Tocilizumab, Sarilumab |
| BRAF Kinase | Vemurafenib, Dabrafenib |
| Sodium Channel (SCN5A) | Lidocaine, Mexiletine, Flecainide |
| Interleukin-17A (IL-17A) | Secukinumab, Ixekizumab |
| Janus Kinase (JAK) | Tofacitinib, Baricitinib, Ruxolitinib |

The systems and methods herein are not limited to the target proteins and reference ligands listed herein, but find use with any suitable pairs of target proteins and reference ligands.

### Systems and Methods

In a first aspect, assay systems and methods are provided utilizing a multicomponent bioluminescent complex, such as a split luciferase (e.g., NANOBIT, NANOTRIP, etc.), in which a first component of the bioluminescent complex is tethered to a target protein and a second component of the bioluminescent complex is tethered to a reference ligand for the target protein. In such systems, binding of the reference ligand to the target protein is required for the first and second components to form an active bioluminescent complex. In typical embodiments, the target protein is a potential drug target, and the reference ligand is a compound, peptide, or other agent with known binding affinity for a potentially druggable location on the target protein. When the reference ligand binds to the target protein, the bioluminescent complex is formed. In the presence of a substrate for the bioluminescent complex, detectable bioluminescence is produced, the level of which correlates to the binding of the reference ligand to the target protein. Upon addition of a test ligand (e.g., candidate drug) for the target protein, the test ligand competes with the reference ligand. Binding of the test ligand to the target protein displaces the reference ligand from the target protein. Because the formation of the bioluminescent complex requires binding of the target protein by the reference ligand, disruption of that binding results in loss of bioluminescent complex and reduction in the detectable bioluminescent signal. The affinity of the test ligand for the target protein can be calculated by determining the concentration dependent reduction in bioluminescent signal.

In particular embodiments of the first aspect, assay systems and methods are provided utilizing the NANOBIT bioluminescent complex reporter system (or a variant thereof), in which the first component of the bioluminescent complex is LGBIT (SEQ ID NO: 5) or a variant thereof (e.g., having at least 70% identity to SEQ ID NO: 5) and the second component of the bioluminescent complex is SMBIT (SEQ ID NO: 4) or a variant thereof (e.g., having at least 70% identity to SEQ ID NO: 4). The SMBIT peptide (or variant thereof) is tethered to the reference ligand and the LGBIT polypeptide (or variant thereof) is tethered to the target protein. As described herein the tethering of the components can be by any suitable linkage, including but not limited to antibody-affinity tag, fusion, HALOTAG-HT ligand, biotin-streptavidin, chemical linker, etc. Because SMBIT and LGBIT have low affinity for each other in the absence of external facilitation, binding of the reference ligand to the target protein is required to facilitate NANOBIT complex formation and to produce above-background bioluminescence in the presence of a suitable substrate (e.g., a coelenterazine, a furimazine, etc.).

In a second aspect, assay systems and methods are provided utilizing a bioluminescent protein (e.g., NANOLUC) tethered to a target protein and a fluorophore tethered to a reference ligand for the target protein. In such systems, binding of the reference ligand to the target protein brings the fluorophore within proximity of the bioluminescent protein, thereby allowing BRET from the bioluminescent protein to the fluorophore. In typical embodiments, the target protein is a potential drug target, and the reference ligand is a compound, peptide, or other agent with known binding affinity for a potentially druggable location on the target protein. In the presence of a substrate for the bioluminescent protein, detectable fluorescence is produced via BRET from the donor bioluminescent protein to the acceptor fluorophore, the level of which correlates to the binding of the reference ligand to the target protein. Upon addition of a test ligand (e.g., candidate drug) for the target protein, the test ligand competes with the reference ligand. Binding of the test ligand to the target protein displaces the reference ligand from the target protein. Because BRET in this system requires close proximity of the fluorophore and bioluminescent protein resulting from binding of the target protein by the reference ligand, disruption of that binding results in loss of BRET and reduction in the detectable fluorescent signal. The affinity of the test ligand for the target protein can be calculated by determining the concentration dependent reduction in BRET/fluorescent signal.

In particular embodiments of the second aspect, assay systems and methods are provided utilizing the NANOLUC bioluminescent protein (SEQ ID NO: 1) or a variant thereof (e.g., having at least 70% identity to SEQ ID NO: 1). The NANOLUC bioluminescent protein (or variant thereof) is tethered to the target protein and a fluorophore with an excitation spectrum overlapping the emission spectrum of NANOLUC bioluminescent protein (or variant thereof) is tethered to the reference ligand. As described herein the tethering of the components can be by any suitable linkage, including but not limited to antibody-affinity tag, fusion, HALOTAG-HT ligand, biotin-streptavidin, chemical linker, etc. Because BRET is typically limited to donors and acceptors within 10 nm of each other, binding of the reference ligand to the target protein is required to produce a BRET signal (fluorescence from the fluorophore resulting from excitation by the bioluminescent signal of the NANOLUC protein). If the test ligand competes with and displaces the reference ligand on the target protein, the BRET signal is decreased.

In a third aspect, assay systems and methods are provided utilizing a multicomponent bioluminescent complex, such as a split luciferase (e.g., NANOBIT, NANOTRIP, etc.), in which a first component of the bioluminescent complex is tethered to a target protein, a second component of the bioluminescent complex is untethered, and a fluorophore with an excitation spectrum overlapping the emission spectrum of the bioluminescent complex (or variant thereof) is tethered to the reference ligand. In such systems, the components of the bioluminescent complex have high affinity for one another, thereby allowing formation of the bioluminescent complex without facilitation by an external binding event. Binding of the reference ligand to the target protein brings the fluorophore within proximity of the bioluminescent complex, thereby allowing BRET from the bioluminescent complex to the fluorophore. In typical embodiments, the target protein is a potential drug target, and the reference ligand is a compound, peptide, or other agent with known binding affinity for a potentially druggable location on the target protein. In the presence of a substrate for the bioluminescent protein, detectable fluorescence is produced via BRET from the donor bioluminescent complex to the acceptor fluorophore, the level of which correlates to the binding of the reference ligand to the target protein. Upon addition of a test ligand (e.g., candidate drug) for the target protein, the test ligand competes with the reference ligand. Binding of the test ligand to the target protein displaces the reference ligand from the target protein. Because BRET in this system requires close proximity of the fluorophore and bioluminescent complex resulting from binding of the target protein by the reference ligand, disruption of that binding results in loss of BRET and reduction in the detectable fluorescent signal. The affinity of the test ligand for the target protein can be calculated by determining the concentration dependent reduction in BRET/fluorescent signal.

In particular embodiments of the first aspect, assay systems and methods are provided utilizing the NANOBIT bioluminescent complex reporter system (or a variant thereof), in which the first component of the bioluminescent complex is LGBIT (SEQ ID NO: 5) or a variant thereof (e.g., having at least 70% identity to SEQ ID NO: 5) and the second component of the bioluminescent complex is HIBIT (SEQ ID NO: 3) or a variant thereof (e.g., having at least 70% identity to SEQ ID NO: 3). The HIBIT peptide (or variant thereof) is tethered to the reference to the target protein and the LGBIT polypeptide is not tethered to another component of the system. As described herein the tethering of the components can be by any suitable linkage, including but not limited to antibody-affinity tag, fusion, primary/secondary antibody (or antibody fragment), HALOTAG-HT ligand, biotin-streptavidin, chemical linker, etc. Because HIBIT and LGBIT have high affinity for each other, external facilitation of complex formation is not required to facilitate bioluminescent complex formation and to produce above-background bioluminescence in the presence of a suitable substrate (e.g., a coelenterazine, a furimazine, etc.).

### EXPERIMENTAL

### Example 1

Experiments were conducted during development of embodiments herein to demonstrate the utility of an exemplary engagement assay for BTK kinase.

The experiments conducted using this system demonstrated specific luminescence and competitive loss of signal when known BTK binding compound CTx-0294885 was added to the system. An IC₅₀ was calculated for this compound that agreed with the published affinity of the compound.

### Example 2

### CDK8/Cyclin C-His Assay

Experiments were conducted during development of embodiments herein to demonstrate the utility of an exemplary engagement assay for CDK8 kinase.

The experiments conducted using this system provided determination of optimal protein and tracer concentrations. A two-fold serial dilutions of CDK8 (ThermoFisher Cat. No. PV4402) in Kinase Buffer (50 mM HEPES, 7.5, 10 mM MgCl₂, 1 mM EGTA) were made to obtain a 3× stock (i.e., 80 nM final=240 nM dilution) leaving the last tube at OnM CDK8. A 3-fold serial dilutions of SmBiT Tracer in Kinase Buffer were made to obtain 3× stocks (i.e., 100 nM final=300 nM) with the last tube at OnM Tracer. Anti-HIS-LgBiT was diluted in 1× Lumit® Buffer A to a 3× stock. Dilutions have been successful at 1:200 and 1:400 final dilutions (1:66 and 1:130 as 3× stocks). 5 ul each of 3×CDK8, 3× Tracer (pipetting right to left), and 3× anti-HIS-LgBiT (pipetting bottom to top) were added to wells of a white, LoBind 384-well plate, and incubate with shaking at 500 rpm for 60 min.

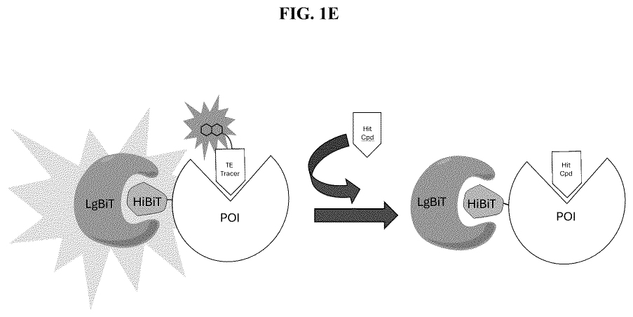

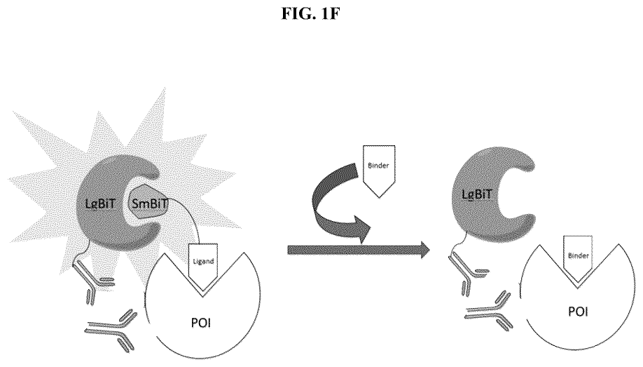

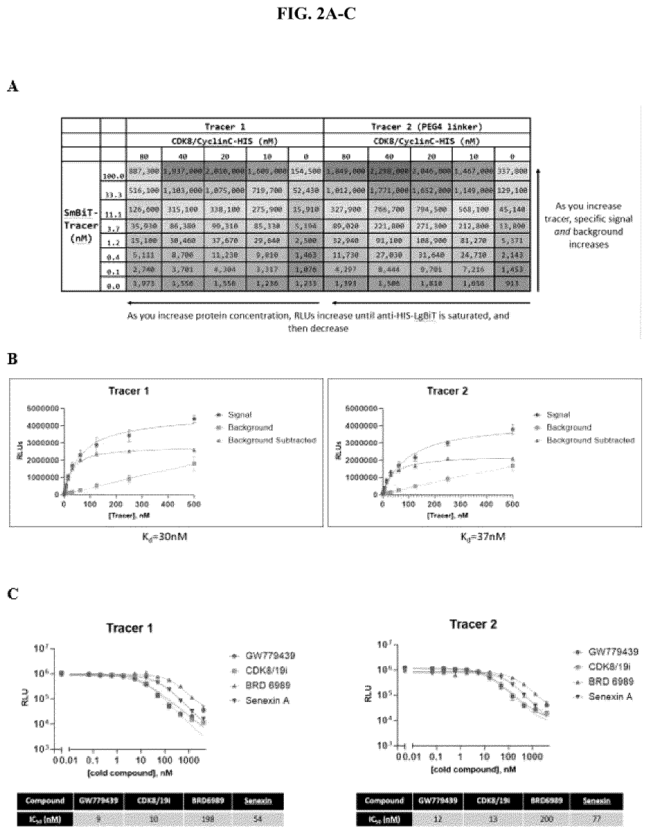

NANO-GLO Luciferase Assay Substrate was diluted 1:50 in Lumit® Buffer A and pipetted into a reservoir, 5 ul added to each well (pipetting bottom to top), and mixed briefly at 500 rpm. Luminescence was read after −2 min. Protein and tracer concentrations (FIG. 2A), Kd of the CDK8-tracer (FIG. 2B), and potency rank order of know CDK8 inhibitors (FIG. 2C) was determined.

SEQUENCES

SEQ ID NO: 1-NANOLUC-
MKHHHHHHAIAMVFTLEDFVGDWRQTAGYNLDQVLEQGGVSSLFQNLGV
SVTPIQRIVLSGENGLKIDIHVIIPYEGLSGDQMGQIEKIFKVVYPVDD
HHFKVILHYGTLVIDGVTPNMIDYFGRPYEGIAVFDGKKITVTGTLWNG
NKIIDERLINPDGSLLFRVTINGVTGWRLCERILAV
SEQ ID NO: 2-full-length NANOBIT-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
DGSMLFRVTINSVTGYRLFEEIL
SEQ ID NO: 3-HIBIT-
VSGWRLFKKIS
SEQ ID NO: 4-SMBIT-
VTGYRLFEEIL
SEQ ID NO: 5-LGBIT-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
DGSMLFRVTINSHHHHHH
SEQ ID NO: 6-LgTrip-
MVFTLEDFVGDWEQTAAYNLDQVLEQGGVSSLLQNLAVSVTPIQRIVRS
GENALKIDIHVIIPYEGLSADQMAQIEEVFKVVYPVDDHHFKVILPYGT
LVIDGVTPNMLNYFGRPYEGIAVFDGKKITVTGTLWNGNKIIDERLITP
D
SEQ ID NO: 7-SmTrip9-
GSMLFRVTINS
SEQ ID NO: 8-HALOTAG-
MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRTTDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISG

1. A system comprising:
(a) a bioluminescent protein or a bioluminescent complex tethered to a target protein;
(b) fluorophore tethered to a reference ligand for the target protein;
(c) a test ligand for the target protein; and
(d) a substrate for the bioluminescent protein or complex;
wherein the bioluminescent protein or complex emits a bioluminescent signal at a first wavelength in the presence of the substrate;
wherein the fluorophore absorbs light at the first wavelength and emits a fluorescent signal at the second wavelength;
wherein when the reference ligand is bound to the target protein the fluorophore is in proximity to the bioluminescent protein or complex to allow bioluminescence resonance energy transfer (BRET) from the bioluminescent protein or complex; and
wherein if the test ligand is capable of binding to the target protein the test ligand competes with the reference ligand for binding to the target protein, thereby decreasing the fluorescent signal resulting from the BRET.

2-3. (canceled)

4. The system of claim 1, wherein the bioluminescent protein is an Oplophorous-derived polypeptide that comprises at least 70% sequence identity with SEQ ID NO: 1.

5. (canceled)

6. The system of claim 1, wherein the bioluminescent protein is a circularly permuted variant of an Oplophorous-derived polypeptide.

7. The system of claim 1, wherein the bioluminescent protein or a bioluminescent complex tethered to a target protein is a bioluminescent complex; wherein a first component of the bioluminescent complex is tethered to the target protein; wherein a second component of the bioluminescent complex is not tethered to the target protein.

8. The system of claim 7, wherein the first and second components of the bioluminescent complex form an active bioluminescent complex upon binding to each other; and wherein the first and second components of the bioluminescent complex require facilitation to form an active bioluminescent complex.

9. The system of claim 8, wherein the first and second components of the bioluminescent complex collectively comprise at least 70% sequence identity with SEQ ID NO: 1 or 2.

10. The system of claim 9, wherein the first component of the bioluminescent complex comprises at least 70% sequence identity with SEQ ID NO: 3.

11. (canceled)

12. The system of claim 10, wherein the second component of the bioluminescent complex comprises at least 70% sequence identity with SEQ ID NO: 5.

13. The system of claim 1, wherein the bioluminescent protein or complex is fused to the target protein directly or by a linker peptide/polypeptide.

14. The system of claim 1, wherein the bioluminescent protein or complex is tethered to an affinity molecule capable of binding to the target protein or an affinity tag thereon.

15. The system of claim 14, wherein the affinity molecule is an antibody or antibody fragment, and the target protein is tethered to an affinity tag to which the antibody or antibody fragment is capable of binding.

16. The system of claim 1, wherein the bioluminescent protein or complex is fused a capture agent capable of covalently binding to a capture ligand tethered to the target protein.

17. The system of claim 14, wherein the capture agent is a modified dehalogenase capable of forming a covalent bond to a haloalkyl moiety and the capture ligand is a haloalkyl moiety and wherein the modified dehalogenase comprises at least 70% sequence identity to SEQ ID NO: 8.

18. (canceled)

19. The system of claim 1, wherein the bioluminescent protein or complex is tethered to a secondary affinity molecule capable of binding to a primary affinity molecule capable of binding to the target protein or an affinity tag thereon.

20. The system of claim 1 wherein the substrate for the bioluminescent protein or complex is a coelenterazine molecule.

21. (canceled)

22. The system of claim 20, wherein the coelenterazine molecule is furimazine or fluorofurimazine.

23. (canceled)

24. The composition of claim 1, wherein the fluorophore is a rhodamine dye selected from:

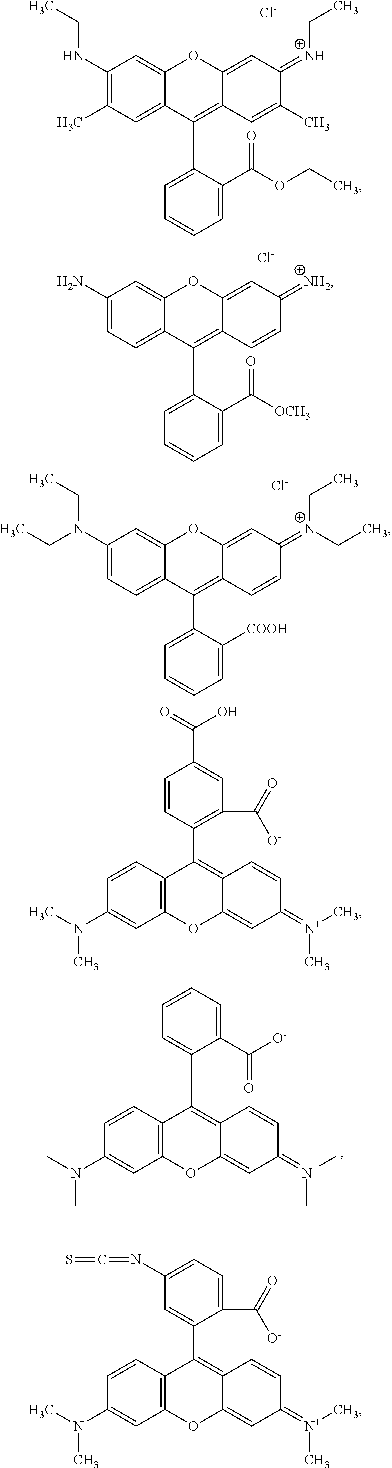

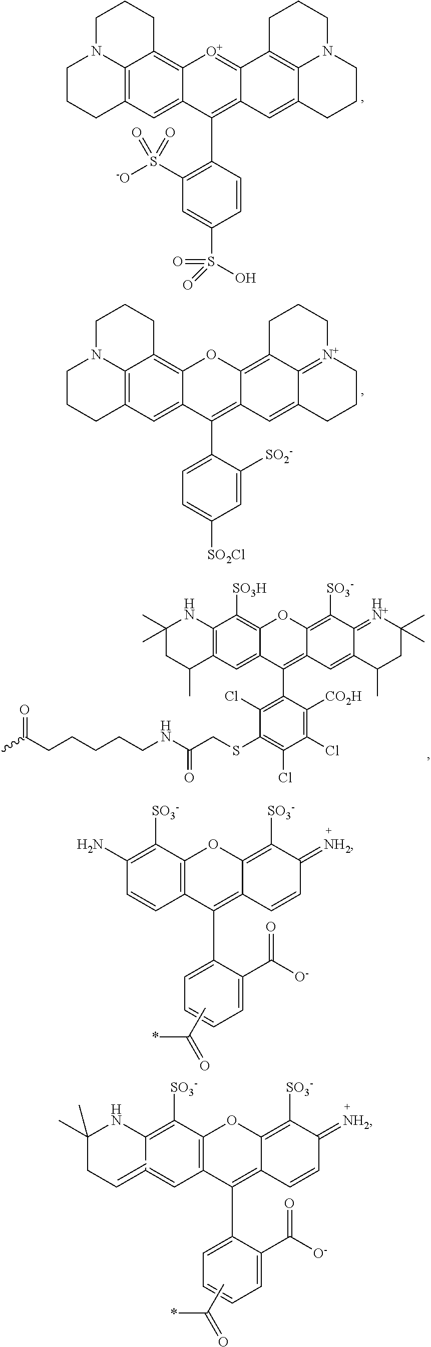

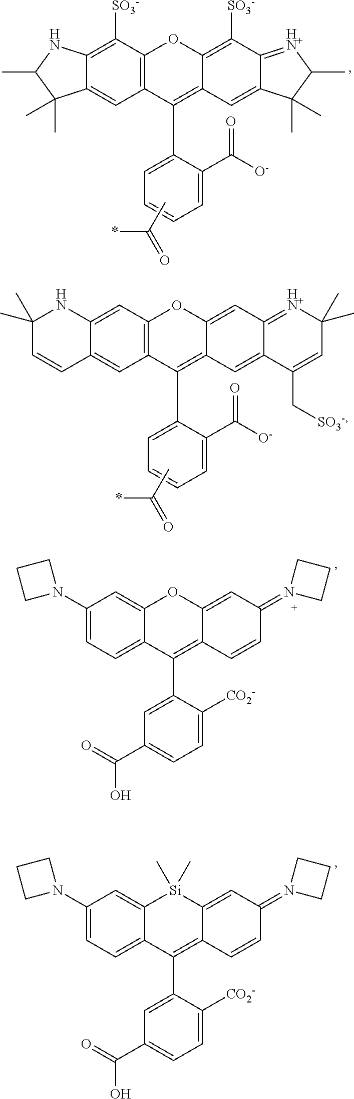

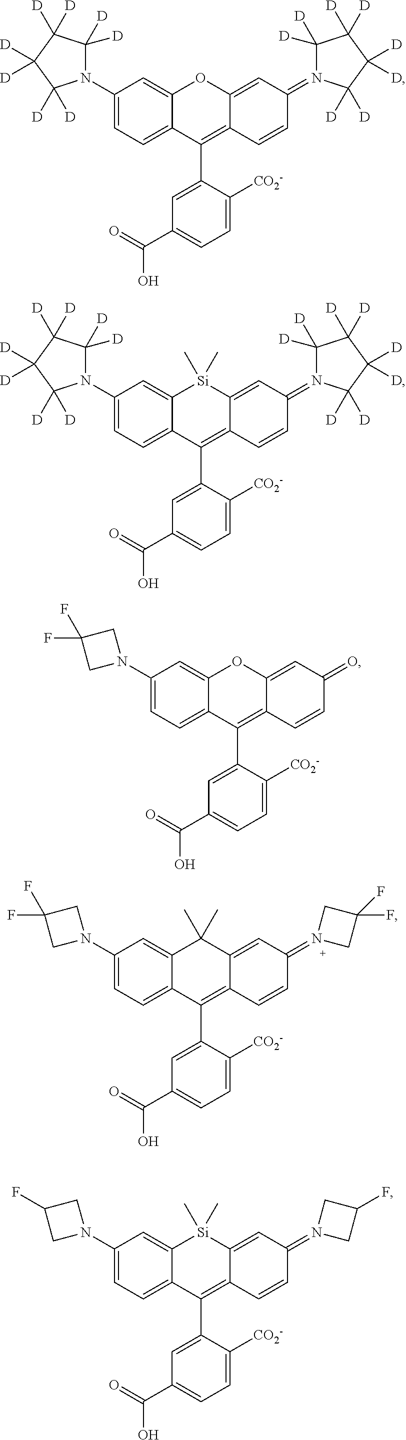

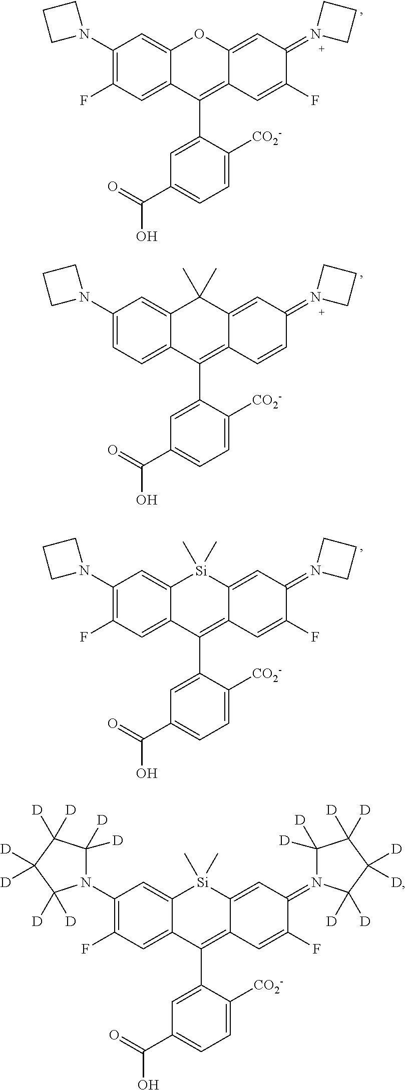

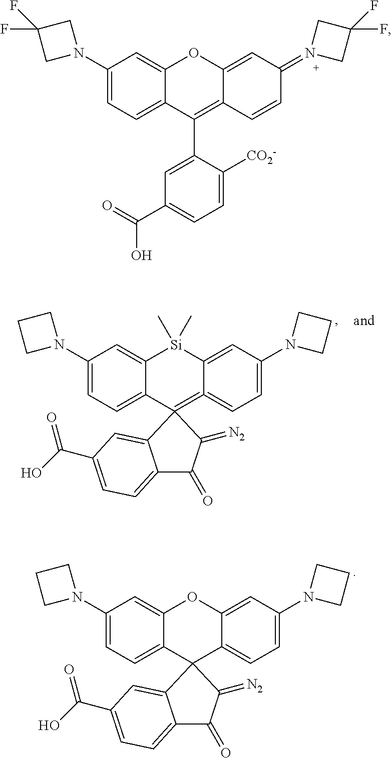

25. A system comprising:
(a) a first component of a bioluminescent complex tethered to a target protein;
(b) a second component of the bioluminescent complex tethered to a reference ligand;
(c) a test ligand for the target protein; and
(d) a substrate for the bioluminescent protein or complex;
wherein the bioluminescent protein or complex emits a bioluminescent signal in the presence of the substrate;
wherein when the reference ligand is bound to the target protein the second component of the bioluminescent complex is in proximity to the first component of the bioluminescent complex to facilitate formation the bioluminescent complex and generation of the bioluminescent signal in the presence of the substrate; and
wherein if the test ligand is capable of binding to the target protein the test ligand competes with the reference ligand for binding to the target protein, thereby decreasing the bioluminescent signal from the bioluminescent complex.

26-41. (canceled)

42. A method comprising:
(a) contacting within a biochemical sample:
(i) a bioluminescent protein or a bioluminescent complex tethered to a target protein;
(ii) fluorophore tethered to a reference ligand for the target protein; and
(iii) a substrate for the bioluminescent protein or complex;
wherein the bioluminescent protein or complex emits a bioluminescent signal at a first wavelength in the presence of the substrate;
wherein the fluorophore absorbs light at the first wavelength and emits a fluorescent signal at the second wavelength;
wherein when the reference ligand is bound to the target protein the fluorophore is in proximity to the bioluminescent protein or complex to allow bioluminescence resonance energy transfer (BRET) from the bioluminescent protein or complex;
(b) detecting bioluminescence from the bioluminescent protein or complex;
(c) contacting the sample with a test ligand for the target protein; and
(d) detecting bioluminescence from the bioluminescent protein or complex;
wherein if the test ligand is capable of binding to the target protein the test ligand competes with the reference ligand for binding to the target protein, thereby decreasing the fluorescent signal resulting from the BRET.

42-65. (canceled)

66. A method comprising:
(a) a first component of a bioluminescent complex tethered to a target protein;
(b) a second component of the bioluminescent complex tethered to a reference ligand;
(c) a test ligand for the target protein; and
(d) a substrate for the bioluminescent protein or complex;
wherein the bioluminescent protein or complex emits a bioluminescent signal in the presence of the substrate;
wherein when the reference ligand is bound to the target protein the second component of the bioluminescent complex is in proximity to the first component of the bioluminescent complex to facilitate formation the bioluminescent complex and generation of the bioluminescent signal in the presence of the substrate; and
wherein if the test ligand is capable of binding to the target protein the test ligand competes with the reference ligand for binding to the target protein, thereby decreasing the bioluminescent signal from the bioluminescent complex.

67-82. (canceled)


SEQUENCE LISTING (8 sequences)

SEQ ID NO: 1 - protein, 183 aa, Synthetic construct
MKHHHHHHAIAMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAV

SEQ ID NO: 2 - protein, 170 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSVTGYRLFEEIL

SEQ ID NO: 3 - protein, 11 aa, Synthetic construct
VSGWRLFKKIS

SEQ ID NO: 4 - protein, 11 aa, Synthetic construct
VTGYRLFEEIL

SEQ ID NO: 5 - protein, 165 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINSHHHHHH

SEQ ID NO: 6 - protein, 148 aa, Synthetic construct
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPD

SEQ ID NO: 7 - protein, 11 aa, Synthetic construct
GSMLFRVTINS

SEQ ID NO: 8 - protein, 297 aa, Synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG